(19) United States
(12) Patent Application Publication
(10) Pub. No.: US 2012/0064011 A1
(43) Pub. Date: Mar. 15, 2012

(54) PREPARATION FOR EXTERNAL APPLICATION

(75) Inventor: Dirk Schumann, Leipzig (DE)

(73) Assignee: Bubbles and Beyond GmBH, Leipzig (DE)

(21) Appl. No.: 13/257,100

(22) PCT Filed: Mar. 19, 2010
(86) PCT No.: PCT/EP2010/001742

§ 371 Date: Nov. 29, 2011

(30) Foreign Application Priority Data
Mar. 19, 2009 (DE) 10 2009 013 469.7

(51) Int. Cl. A61K 49/00; A61K 47/14; A61K 47/16; A61K 47/44; A61K 47/08; A61K 47/20; A61K 47/24; A61K 8/34; A61K 8/37; A61K 8/35; A61K 8/44; A61K 8/46; A61K 8/55; A61K 8/92; A61Q 19/00; A61P 23/00; A61K 47/10; B82Y 5/00

(52) U.S. Cl. 424/9.8; 514/772; 514/785; 514/784; 977/700; 977/906

(57) ABSTRACT

Preparations for external application to human and animal skin, comprising: a) a composition in the form of a fluid nanophase system, comprising the components of a1) at least one water-insoluble substance with a water solubility of less than 4 gram per liter, a2) at least one amphiphilic substance (NP-MCA), which has no surfactant structure, does not build structures alone, the solubility of which is between 4 g and 1000 g per liter in water or oil and which does not enrich preferably at the oil-water interface, a3) at least one anionic, cationic, amphoteric and/or non-ionic surfactant, a4) at least one polar protic solvent, in particular having hydroxy functionality, a5) if necessary one or more adjuvants, wherein the percentage relate to the total weight of the composition each; and b) a therapeutic, cosmetic or diagnostically effective agent in a therapeutic, cosmetic or diagnostically effective amount.

### CROSS REFERENCE TO RELATED APPLICATIONS

This application is the U.S. National Phase Application of PCT/EP2010/001742, filed Mar. 19, 2010, which claims priority to German Patent Application No. 10 2009 013 469.7, filed Mar. 19, 2009, the contents of such applications being incorporated by reference herein.

### FIELD OF THE INVENTION

The present invention relates to preparations for external application to human and animal skin. In particular, the present invention relates to preparations which are suitable for transporting an active ingredient for the purpose of a therapeutic or non-therapeutic or cosmetic treatment into the skin (intradermal) or through the skin (transdermal) of animals and humans. Quite particularly, the present invention relates to preparations for improving the absorption, penetration or permeation of a pharmaceutically, cosmetically or diagnostically active agent into the skin, or the transport of a pharmaceutically, cosmetically or diagnostically active agent through the skin. Furthermore, the present invention relates to the use of such preparations, methods for their preparation, their applications and devices, as well as methods for achieving a non-systemic intradermal or systemic transdermal effect.

### BACKGROUND OF THE INVENTION

The skin of animals and humans not only has a mechanical function as a boundary layer between the inside of the body and the outside world, but in particular also fulfils a protective function by forming a barrier against the penetration of harmful substances and sealing the body off against them.

In addition to the administration of active ingredients to the human or animal body for therapeutic or non-therapeutic purposes by mouth, by injection or by infusion, it has proved advantageous if such substances are transported into the skin for topical application or through the skin for systemic distribution in the body, as these application routes may represent a method of administering active ingredients which has fewer side-effects. This means that the gastrointestinal route or passage through the liver, during which many active ingredients are deactivated or metabolized, is by-passed, or flooding with a high concentration of the active ingredients is avoided. The advantages which result from such an application route for active ingredients have been thoroughly investigated and are long known.

Both in the case of the topical application of active ingredients onto the skin and in the case of systemic administration of active ingredients through the skin, there is basically a problem in that the absorption or penetration of active ingredients which are to enter the skin or be transported through the skin are opposed by the already mentioned natural barrier, which is caused in particular by the top layer of the epidermis, the stratum corneum.

Because of the lipophilic character of the skin, the mostly water-soluble active ingredients are not absorbed by the skin at all, or only with difficulty.

In order to effectively transport pharmaceutical, cosmetic or diagnostic active ingredients from the outside into or through the skin, agents which act as penetration improver or permeation improvers which overcome this protective barrier and modify the absorbency or permeability of the skin so as to make it easier to transport active ingredients into or through the skin or actually make it possible in the first place are advantageous.

Various plasters which are intended in particular to transdermally transport an active ingredient to be applied have been developed. However, the main drawback of these techniques is that they cause skin irritations or that there are application-related and economic limits to the size of such plasters for increasing surface contact.

In order to improve the permeability of the skin for active ingredients it is also known to combine the relevant active ingredient with a penetration improver or a permeability improver.

For these purposes, the use of oils (WO 93/12744 A1), which is incorporated by reference, proteases (WO 2001/087255), which is incorporated by reference, combinations of a fatty alcohol, a diethylene glycol monoalkyl ether and a carrier consisting of water, a C1-C4 alcohol and a polyalcohol (WO 2002/011768 A1), which is incorporated by reference, combinations of 2-ethyl-1,3-hexanediol and oleic acid (WO 87/03490), which is incorporated by reference, morpholine or piperazine derivatives (EP 0 268 222 A1), which is incorporated by reference, or higher n-alkane carboxylic acids (EP 0305726 A1), which is incorporated by reference, have been proposed.

However, all these proposals have the disadvantage, that they make use of systems and substances which are either costly in technical terms, dermatologically or allergologically problematical or can be used only for specific active ingredients.

Furthermore, it is a fact that the efficacy of a formulation of a medicinal product to be used topically, intradermally or transdermally cannot be predicted for every active ingredient and can prove to be very different, as it depends on a very wide variety of physiological, physical and chemical conditions and properties, such as for example, condition of the skin, interactions of the active ingredients with an agent acting as a penetration improver or permeability improver and the tissue, or the chemical nature of the active ingredient.

A universally usable topical preparation for all active ingredients that is to be applied dermally for transport into or through the skin is not known from the state of the art.

In this connection, reference is made to the statements on this subject in European patent application EP 1323430 A2, paragraphs [0002] to [0023], which is incorporated by reference herein.

### SUMMARY OF THE INVENTION

Therefore, there is still a need for percutaneous penetration-enhancing or skin permeability-increasing agents or preparations which are capable of transporting an active ingredient into or through the skin efficiently and without undesired side-effects. In general, an object of the invention is to remove the disadvantages underlying the state of the art.

Against the background of the state of the art, an object of the present invention is in particular to provide a preparation or a composition which makes possible a transdermal transportation of active ingredient through the skin without having the disadvantages of the state of the art.

A further object of the present invention is the provision of a preparation or composition which makes possible an intradermal transportation of active ingredients into the skin without fully penetrating it, and without having the disadvantages of the state of the art.

An object of the invention is also the use of a preparation, optionally for producing a medicinal product or cosmetic agent, for the transportation of a therapeutically or cosmetically active agent into or through the skin of a human or animal.

Furthermore, an object of the present invention is to provide a method for producing an application system for the therapeutic or non-therapeutic, i.e. cosmetic, treatment of the human or animal.

In addition, an object of the present invention is the provision of a method for achieving a cosmetic or therapeutic effect in a tissue by application of a preparation to be applied externally.

Finally a further object of the present invention is to provide a method for improving or enhancing the penetration or permeation of a pharmaceutically or non-pharmaceutically, i.e. cosmetically, acting substance through the top layer, in particular the epidermis, of the skin of humans and animals.

Moreover an object of the present invention is to provide a method for producing a preparation for the topical, non-transdermal, or intradermal or transdermal application to the human or animal body.

### BRIEF DESCRIPTION OF THE DRAWINGS

FIG. 1:

Scatter of a green laser beam (Conrad Electronic, Germany, model no. GLP-101, 530-545 nm) to detect the nanostructuring in liquid systems with a) water, b) fluid nanophase system according to aspects of the invention (acting as penetration improver or permeation improver) of the following composition (the given percentages by weight are relative to the complete composition): aqueous phase: water (35.70 wt.-%); oil phase: Cetiol OE (20.03 wt.-%); surfactant: Lutensol TO 3 (11.14 wt.-%), Tween 80 (6.86 wt.-%); sodium cocoyl isethionate (0.42 wt.-%); NP-MCA: triethylcitrate (6.89 wt.-%), 2-ethyl-1,3-hexanediol (17.87 wt.-%), active ingredient: tetracaine hydrochloride (1.09 wt.-%).

FIG. 2:

FIG. 2 shows, by means of a freezefracture electron microscopy picture, the nanostructure of the fluid nanophase system according to aspects of the invention (the composition aqueous phase: water (55.28 wt.-%); oil phase: orange terpene (11.35 wt.-%); surfactant: sodium dodecyl sulphate (8.80 wt.-%), C₉-C₁₁ alcohol ethoxylate (4) (8.82 wt.-%); NP-MCA: diacetone alcohol (3.47 wt.-%), ethyl acetoacetate (12.28 wt.-%) (the given percentages by weight are relative to the complete composition). The smaller spherical structures are micelles of the aqueous phase that are approximately 20-50 nm large and are distributed within an oil phase of small structure.

FIG. 3:

Phase diagram (fish diagram or whale diagram) which represents the course of the single-phase and two-phase and lamellar existence ranges of a fluid nanophase system according to aspects of the invention as a function of the surfactant concentration and the temperature. There are shown in a) a composition (water/orange terpene PEG-7 glyceryl cocoate/Berol 260 with a water-orange terpene ratio of 1 and a proportion of 20 wt.-% Berol 260 in the surfactant mixture of PEG-7 glyceryl cocoate/Berol 260) as microemulsion, and in b) the same composition additionally containing 4 wt.-% NP-MCA (ethyl acetoacetate (EAA)) as fluid nanophase system (the given percentages by weight are relative to the complete composition). The temperature range, ΔT, of the single-phase existence range of the cleaning agent is represented, wherein ΔT is determined by the length ascertained in the fish diagram of the tangent parallel to the temperature axis at the Lα field which is limited by the intersections of the tangent with the lower and upper dividing lines between single-phase and two-phase existence range of the cleaning agent. As can be seen from FIG. 3, the presence of NP-MCA results in an enlargement of the temperature range ΔT.

### DEFINITION OF TERMS

The term “preparation”, unless explicitly used otherwise, covers a physiologically compatible preparation suitable for therapeutic or cosmetic use, for external application in humans and animals, which contains the composition according to aspects of the invention comprising the components as described here, optionally together with customary excipients and carriers, and which contains a therapeutically, cosmetically or diagnostically active agent in a therapeutically or cosmetically or diagnostically active quantity, and which can be applied externally in a manner known per se.

The term “composition”, unless explicitly used otherwise, covers a combination of various components which brings about the improvement or enhancement of the percutaneous penetration or permeation of an active ingredient or mixture of active ingredients to be applied externally and into which one or more therapeutically, cosmetically or diagnostically active agents or substances can be incorporated for intradermal or transdermal application.

By “skin”, unless otherwise indicated, is meant the outer body covering enveloping humans and animals which comprises in particular the cutis typical in vertebrates for protection against external influences consisting of the ectodermal epidermis and the mesodermal connective tissue.

By the term “active ingredient” is meant materials, compounds or substances, which either i) produce a change in a physiological state in or on the body of a human or animal and serve to heal, alleviate, prevent or recognize diseases in or on the human or animal body and are known to a person skilled in the art as pharmaceutically active materials, compounds or substances, individually or in combination, being synonymous with pharmacologically active agents, pharmaceutical, pharmaceutically active agent, therapeutically or diagnostically active agent or which ii) produce a medicament-like effect and which are known to a person skilled in the art as “cosmeceuticals” (e.g. vitamins, enzymes, essential oils, antioxidants), or iii) display none of the known pharmaceutical or medicament-like effects described under i) and ii), are known as cosmetic substances or cosmetics or as cosmetically active agents and are applied in particular for the purpose of improving the appearance of the body, without affecting and changing the physiological state of the body, i.e. its structure and functions (e.g. dyes, UV-filters, lightening agents).

By “therapeutic treatment” is generally meant the treatment of a human or animal with an active ingredient or medicament with the aim of bringing about a change in physiological state for the purpose of healing or alleviating discomfort. Accordingly, “therapeutically effective” or analogous uses of this expression is to be taken to mean that the active ingredient is present in the relevant tissue in a sufficient quantity and produces the desired therapeutic effect.

By “non-therapeutic” treatment is meant within the meaning of the present invention an application of cosmetic substances or cosmetics in humans or animals which does not serve for healing purposes, for example to achieve a visual change in the skin, in particular a change in colour, or protection against solar radiation. Accordingly, “cosmetically effective” or analogous uses of this expression is to be taken to mean that the active ingredient is present on or in the relevant tissue in a sufficient quantity and produces the desired therapeutic effect.

To this extent the term “cosmetic active ingredient”, or terms used synonymously, covers an agent which is suitable for application on the skin, in order to subjectively and objectively positively change the appearance of the skin, which may be reflected for example in a change in skin colour, tanning, skin lightening, the reduction or disappearance of hyperpigmentation or hyperkeratinization or acne or cellulitis.

The terms “penetration improver” or “permeation improver” used here relate to the nature of the function of the composition on which the invention is based, namely the improvement of or increase in the permeability of the skin for an active ingredient for the purpose of a faster and more effective transport of the relevant active ingredient into the skin or through the skin. This effect can for example be demonstrated in vitro or in vivo by suitable equipment and measurements, for example as described in WO 02/011768 A1.

The use of the term “into the skin” is to be taken to mean that the relevant active ingredient enters the skin without essentially fully penetrating it, with the result that the relevant active ingredient is transported intradermally, i.e. very largely remaining in the skin, but not transdermally through the skin, and no or essentially no systemic, i.e. no therapeutically relevant or otherwise active quantities reach the bloodstream, but a predominantly local effect develops at the site of the application. The term “intradermal” is also to be understood in this connection.

By the term “through the skin” is meant that the relevant active ingredient enters the skin and essentially penetrates it fully, with the result that the relevant active ingredient is transported transdermally and thus a therapeutically desired quantity thereof essentially or predominantly passes into the bloodstream, and a systemic effect can be achieved by the absorption of the active ingredient into the bloodstream. The term “transdermal” is to be understood accordingly.

The term “excipients and carriers” is used here for substances which are not essential to the preparation according to aspects of the invention and make no, or no essential, contribution to the improvement or enhancement according to aspects of the invention of the percutaneous penetration or permeation of an externally applied active ingredient or mixture of active ingredients.

Mentions of “apply externally” or “use externally” are to be understood in connection with the preparation according to aspects of the invention to mean the application thereof to the skin of humans and animals in order to transport an active ingredient intradermally into the skin or transdermally through the skin.

Unless explicitly stated otherwise, the quantity data given in percent or the percentage data are in each case relative to the total weight of the composition or preparation.

The present invention achieves the objects set out above and others by providing a preparation, as defined here in the claims, with which pharmaceutical or cosmetic active ingredients can be transported in a large quantity intradermally into the skin, i.e. without passing completely through the skin tissue, or transdermally through the skin, i.e. passing completely through the skin tissue.

However, diagnostically active agents can also, as will be described in detail below, be advantageously transported into the skin or through the skin with the preparation according to aspects of the invention.

The present invention has many advantages vis-à-vis the state of the art of which the following may chiefly be mentioned:

The method for producing the preparation according to aspects of the invention or the composition according to aspects of the invention requires no equipment or machinery which is technically elaborate or susceptible to faults, but can be carried out with a simple stirrer, for example by means of the Unguator® technology familiar to a person skilled in the art or by means of a magnetic stirrer, which is also of great advantage for production on an industrial scale.

Special physical treatments or pre-treatments (for example fusions) or chemical reactions (for example polymerizations or cross-linkings) are also unnecessary.

Furthermore, the present invention offers the advantage that the active ingredients provided for intradermal or transdermal application can be incorporated directly, without prior alteration (for example solubilization or derivatization), with the result that the physicochemical condition of the relevant active ingredient (polar, amphiphilic, zwitterionic, lipophilic active ingredients) is essentially not critical for intradermal or transdermal transport.

Furthermore, it is advantageous that the composition according to aspects of the invention can be stored for a prolonged period, depending on the storage temperature, with the result that in the case of a consecutive application, according to which first the composition and then, after a time delay, the active ingredient is applied, there is no need to fear any disadvantageous interactions between the composition and the active ingredient or any incompatibilities, and no chemically or biologically determined decomposition or degradation methods of the active ingredient in the composition.

Furthermore, the present invention offers the advantage that the nanophase structure of the composition according to aspects of the invention or the preparation according to aspects of the invention is destroyed in the body, with the result that no nanoparticles remain and accumulate in the body.

In addition, the invention has the advantage that, due to prompt destruction of the nanophase structure after the application of the preparation or composition according to aspects of the invention, there is no risk of longer-term undesired and longer-lasting effects or side-effects and no absorption of toxic substances at the application site.

It is also advantageous according to aspects of the invention that the individual nanophase components of the groups oil phase, aqueous phase, NP-MCAs, surfactants and also active ingredients can be exchanged should it turn out that relevant individual substances prove to be incompatible, for example allergenic.

It was surprisingly found that a composition a) which is present in the form of a fluid nanophase system, comprising the components a1) at least one water-insoluble substance with a water solubility of less than 4 grams per litre, a2) at least one amphiphilic substance (NP-MCA) which does not have a surfactant structure, is not structure-forming on its own, the solubility of which in water or oil is between 4 g and 1000 g per litre and which preferably does not accumulate at the oil-water interface, a3) at least one anionic, cationic, amphoteric and/or non-ionic surfactant, a4) at least one polar protic solvent, in particular with hydroxy functionality, a5) optionally one or more excipients is exceptionally suitable for the intradermal and transdermal transportation of a therapeutically, cosmetically or diagnostically active agent.

Completely surprising and not known from the state of the art was the finding that the said composition effects an increase or an improvement of the penetration or permeation of active ingredients to be transported intradermally into the skin or transdermally through the skin. To this extent, the fluid nanophase system according to aspects of the invention acts as a penetration improver or a permeation improver for an active ingredient to be transported into the skin or through the skin.

A subject of the invention is thus a preparation for external application in humans and animals comprising in combination a) a composition in the form of a fluid nanophase system comprising the components

a1) at least one water-insoluble substance with a water solubility of less than 4 grams per litre, in a quantity of from 0.1 to 90 wt.-%,
a2) at least one amphiphilic substance (NP-MCA) which does not have a surfactant structure, is not structure-forming on its own, the solubility of which in water or oil is between 4 g and 1000 g per litre and which preferably does not accumulate at the oil-water interface, in a quantity of from 0.1 to 80 wt.-%,
a3) at least one anionic, cationic, amphoteric and/or non-ionic surfactant; in a quantity of from 0.1 to 45 wt.-%,
a4) at least one polar protic solvent, in particular with hydroxy functionality, in a quantity of between 1.0 and 90 wt.-%,
a5) optionally one or more excipients, in a quantity of from 0.01 to 10 wt.-%, wherein the given percentages are in each case relative to the total weight of the composition,
and
B) at least one therapeutically, cosmetically or diagnostically active agent in a therapeutically, cosmetically or diagnostically active quantity.

A composition of this type is advantageously suitable for the intradermal and transdermal administration of a therapeutically, cosmetically or diagnostically active agent.

Further subjects of the present invention are shown in the following detailed description of the invention.

### DETAILED DESCRIPTION OF THE PREFERRED EMBODIMENTS

### Composition according to the Invention (Fluid Nanophase System)

An object of the present invention is achieved by the provision of a preparation for external application in humans and animals comprising as essential component a composition a) in the form of a fluid nanophase system, which can also be called nanostructured liquid, comprising the components

a1) at least one water-insoluble substance with a water solubility of less than 4 grams per litre, in a quantity of from 0.1 to 90 wt.-%,
a2) at least one amphiphilic substance (NP-MCA) which does not have a surfactant structure, is not structure-forming on its own, the solubility of which in water or oil is between 4 g and 1000 g per litre and which preferably does not accumulate at the oil-water interface, in a quantity of from 0.1 to 80 wt.-%,
a3) at least one anionic, cationic, amphoteric and/or non-ionic surfactant; in a quantity of from 0.1 to 45 wt.-%,
a4) at least one polar protic solvent, in particular with hydroxy functionality, in a quantity of between 1.0 and 90 wt.-%,
a5) optionally one or more excipients, in a quantity of from 0.01 to 10 wt.-%, wherein the given percentages are in each case relative to the total weight of the composition.

The fluid nanophase system according to aspects of the invention advantageously facilitates or supports the percutaneous penetration or permeation of an externally applied active ingredient or mixture of active ingredients into the skin or through the skin and thus makes the relevant active ingredient more effective.

In a preferred embodiment, the fluid nanophase system according to aspects of the invention can comprise at least one further amphiphilic substance with surfactant structure, for example a cosurfactant with hydrophilic-lipophilic molecular proportions.

Multi-component systems of the water, water-insoluble substance (oil), surfactant and optionally cosurfactant type, which form spontaneously and appear as multi-component systems, are known as microemulsions. Microemulsions are thermodynamically stable, nanostructured fluids which consist at least of water or a watery liquid (e.g. glycerol), oil and a surfactant. Microemulsions sometimes also contain cosurfactants and (when ionic surfactants are used) optionally also salts. The structure sizes of the microemulsions most often lie between 10 and 200 nm. Unlike the kinetically stable emulsions or nanoemulsions, the thermodynamically stable microemulsions tend not to cream due to particle coalescence. In microemulsions, larger structures that have formed briefly decompose again into smaller micelles some time later. As a result, microemulsions form of themselves due to their thermodynamic stability, even without thorough mixing. Unlike emulsions, in microemulsions not only do spherical micelles occur, but also elongated micelles (vermicular micelles) and various forms of network-like structures. In the most favourable case, there is a bicontinuous structure in a microemulsion. Here aqueous phase and oil phase permeate via sponge-like interfaces comprising surfactants and optionally cosurfactants.

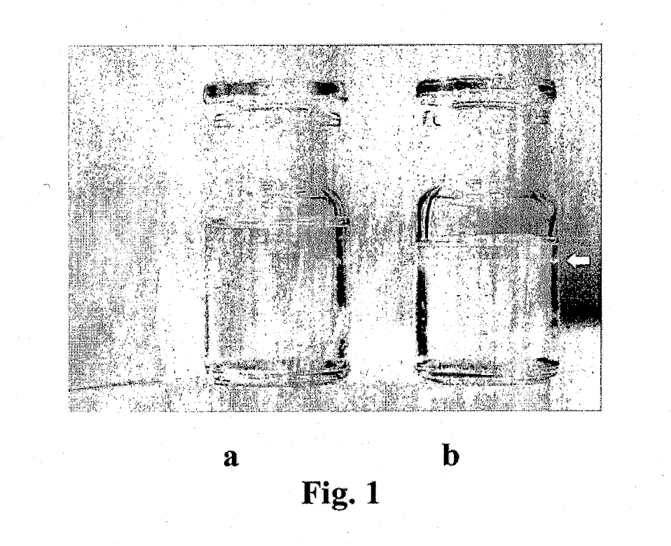

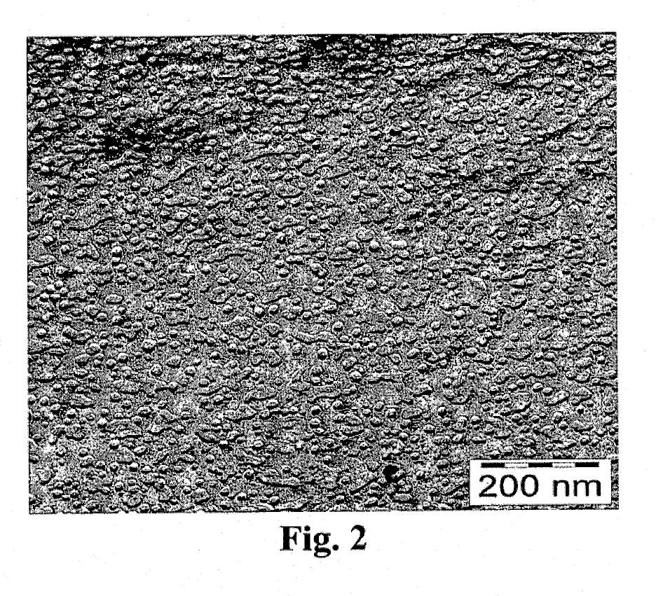

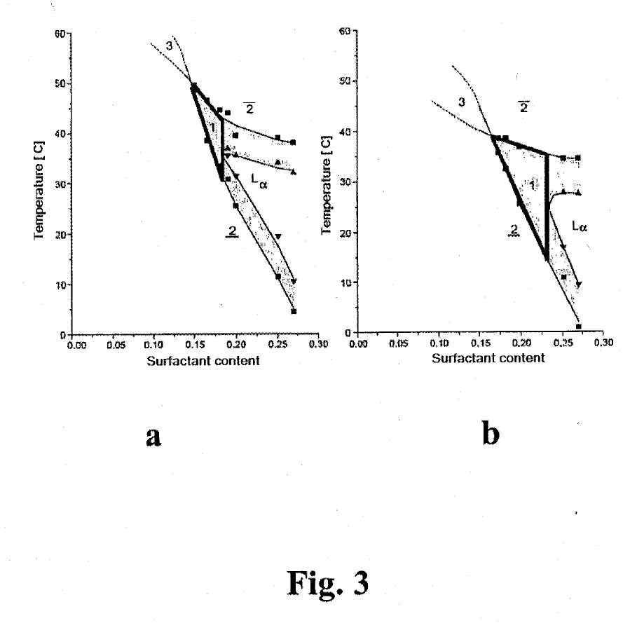

The addition according to aspects of the invention of at least one amphiphilic substance, the so-called NP-MCA (nanophase-forming mixed-chain structure amphiphile), which does not conform to the hydrophilic-hydrophobic structure or properties of surfactant or cosurfactant, makes it possible to advantageously achieve an expansion of the single-phase colloid-disperse area of the microemulsion and to actuate a modification of the properties of the fluid nanophase system, as represented in FIGS. 1 to 3 and described in more detail below.

Surprisingly, it was furthermore ascertained that the addition of NP-MCAs brings about an expansion of the thermodynamically stable, single-phase existence range of the nanostructured systems. This was all the more surprising as the specialists had previously assumed that, the more different the lipophilic and the hydrophilic parts are in respect of their solubility in the respective opposite phase, the more easily microemulsions can form.

Therefore, to produce so-called microemulsions a person skilled in the art used mainly oils and hydrophilic constituents which dissolve in each other as little as possible. Consequently, according to the state of the art, substances which are not surface active and yet remain both in the oil phase and in the hydrophilic phase, as is the case with the non-structure-forming, mixed-chain structure amphiphiles (NP-MCAs) according to aspects of the invention were not used to produce microemulsions.

In this respect, aspects of the present invention also overcome a deep-rooted prejudice among the specialists.

It was furthermore surprising that the addition of NP-MCAs to an oil/water/surfactant mixture allows a clear broadening of the single-phase range of the nanophase fluids that have formed compared with conventional microemulsions to form and, compared with conventional microemulsions, the lamellar phase (La) is largely suppressed in a phase diagram called fish diagram or “whale diagram”, with the result that the occurrence of highly viscous lamellar phases in which the oil and water domains are disadvantageously present in layers is prevented or at least reduced (see FIG. 3).

It was also surprising that the addition according to aspects of the invention of an NP-MCA, for example an ethyl acetoacetate, results in a lowering of the temperature window and thus a larger usable temperature range compared with conventional microemulsions (see FIG. 3) can be achieved.

Within the meaning of the present invention, these systems are called fluid nanophase systems (for short: nanophase fluids). Nanophase fluids contain in particular water or a watery substance, oil, at least one structure-forming amphiphile which adsorbs on the oil-water interface and—expanding to the microemulsions—at least one non-structure-forming amphiphile without surfactant structure (NP-MCA). The structure-forming amphiphile is selected from the group consisting of surfactants, cosurfactants or surfactant-like oligomers or polymers.

The NP-MCAs are important for the expansion of the thermodynamically stable existence range of the fluid nanophases and therefore a further delimitation criterion for the microemulsions. The addition of NP-MCAs advantageously makes possible a clear broadening and optionally lowering of the temperature window of the single-phase range.

It is furthermore advantageous that the NP-MCAs can additionally prevent or reduce the occurrence of highly viscous lamellar phases. Additionally, the NP-MCAs can reduce the surfactant concentration that may be necessary.

In addition, it is also advantageous that the NP-MCAs are able to greatly expand the properties and application possibilities of the nanophase fluids for the transportation of therapeutically or cosmetically active agents.

The group of the nanophase-forming mixed-chain structure amphiphiles (NP-MCAs) comprises mixed-chain structure amphiphiles which have hydrophilic and hydrophobic molecular areas which lie spatially close together but are mixed such that they do not have a surfactant-like structure. They thus differ from surfactants and cosurfactants which obtain their function through the directional separation of the two areas (head-to-tail structure). As a result, NP-MCAs are not capable of forming superlattices on their own and preferably do not accumulate at the oil-water interface. Therefore, besides the oil or aqueous phase, another surfactant is additionally necessary for the formation of nanophase fluids. However, NP-MCAs have a significant solubility in the aqueous phase or oil phase and disperse in the latter until an equilibrium is reached. The solubility of the NP-MCA in water or oil is normally between 4 and 1000 grams per litre, optionally also in the form of its salts.

An NP-MCA according to aspects of the invention comprises an amphiphilic substance which does not have a directional hydrophilic-hydrophobic surfactant structure, is not structure-forming, i.e. not micelle-forming, on its own, the solubility of which in water or oil is between 4 g and 1000 g per litre and which preferably does not accumulate at the oil-water interface.

In microemulsions, a triangle can be stretched between the X-point and the intersection points of the boundary area between the single-phase and the two-phase area and the tangent laid parallel to the Y-axis of the starting La-field in the phase diagram as a function of temperature and surfactant concentration (fish or whale diagram). Measuring methods for constructing the surfactant concentration-temperature phase diagram (fish or whale diagram) are known to a person skilled in the art from the state of the art. NP-MCAs unexpectedly and advantageously result in a broadening of the existence range of the single-phase area, as well as in an enlargement of the surface area of this triangle, and can be defined via this. Preferably all amphiphiles which, if added at 4% to an oil-water-surfactant system, result in an enlargement of the surface area of this triangle by at least 5%, without modifying the surfactant system, preferably by at least 10% and quite particularly preferably by at least 20%, can be used as NP-MCAs. In a particular embodiment, the surface area of the triangle is enlarged in a range of from 5% to 2000%, without modifying the surfactant system, preferably from 10% to 1000%, quite particularly preferably from 15% to 500%.

Particularly preferred are NP-MCAs which are characterized in that, when added to an oil-water-surfactant system containing the constituents oil a1), surfactant a3) and protic solvent a4), and optionally excipients a5) at 4 wt.-% relative to the total weight of the system, they result in an at least 5% enlargement of the surface area of the triangle contained in the phase diagram which is determined by the three corner points:

### i) the X-point,

ii) the upper intersection point of the boundary area between the single-phase and the two-phase area with the tangent laid parallel to the temperature Y-axis at the starting La-field and
iii) the lower intersection point of the boundary area between the single-phase and the two-phase area with the tangent laid parallel to the temperature Y-axis at the starting La-field.

The position of such triangles is illustrated in FIG. 3.

The methodology for constructing such phase diagrams is described for example in:—M. Kahlweit, R. Strey, D. Haase, H. Kunieda, T. Schmeling, B. Faulhaber, M. Borkovec, H. F. Eicke, G. Busse, F. Eggers, T. Funck, H. Richmann, L. Magid, O, Soderman, P. Stilbs, J. Winkler, A. Dittrich, and W. Jahn: “How to Study Microemulsions”, J. Colloid Interf. Sci., 118 (2), 436 (1987)—Microemulsions, T. Sottmann and R. Strey in Fundamentals of Interface and Colloid Science, Volume V, edited by J. Lyklema, Academic Press (2005).

In order to obtain a phase diagram (whale diagram) samples are made up with a constant ratio of the non-surfactant components and a surfactant proportion which is increased stepwise starting from 0% up to a desired surfactant proportion (optionally up to 100%). The step increase is based on the demands of measurement precision, wherein a step size of 2% is most often sufficient. These samples are left in a thermostatted medium (preferably water, possibly with freezing-point-lowering additives) at temperatures of from minus (−) 30° C. to plus (+) 100° C. until the phase balance is established, after which the phase state is assessed visually via the light scatter. The size of the temperature steps results from the desired measurement precision, wherein a step size of 1° C. is most often sufficient for technical applications. The phase boundaries result from the transition from one phase state into the next, wherein the error is predetermined by the step size of the temperature measurement. The thus-obtained measurement points are plotted in a diagram and joined up, wherein the temperature is plotted against the surfactant proportion. In most cases it is enough to find the phase states existing in the measurement range in a sample and to determine the phase boundaries via nested intervals. The value for the phase broadening of the nanostructured fluid composition is determined by presenting a triangle in the phase diagram of FIG. 3, in such a way that a first straight line a) is formed starting from the X-point to the curve characterizing the phase state above the average temperature (line above 2), a second straight line b) is formed such that it tangentially touches the aperture angle of La and the first straight line a) intersects at the site of its tangential contact point with the curve characterizing the curve above the average temperature (line above 2), and a third straight line c) is laid on the curve characterizing the phase state below the average temperature (line below 2) such that it cuts the two straight lines a) and b). A numerical value A1 results from totaling the lengths of the three straight lines in FIG. 3, which corresponds to a microemulsion according to the state of the art. The analogous totaling of the lengths of the straight lines of a phase diagram according to aspects of the invention (nanophase fluid) results in a numerical value A2. The numerical value of the advantageous phase broadening achieved by the present invention is ascertained by forming the A2/A1 ratio, thus by dividing A2 by A1. For the composition according to aspects of the invention of the nanophase fluid, this numerical value is greater than 1.0; particularly, greater than 1.1; in particular, greater than 1.15; quite particularly, greater than 1.2; preferably greater than 1.22. The scale of the triangle can be influenced in addition or alternatively to the enlargement of the surface area of the triangle. Preferred NP-MCAs are characterized in that, when added at 4 wt.-% relative to the total weight of the composition a) according to aspects of the invention to an oil-water-surfactant system containing the constituents a1), a3) and a4), they result in an at least 5% enlargement of the temperature range ΔT of the single-phase existence range of the composition a) according to aspects of the invention, which is determined by the length, ascertained in the phase diagram as a function of temperature and surfactant concentration, of the tangent parallel to the temperature axis at the La field which is limited by the intersection points of the tangent with the lower and upper dividing lines between single-phase and two-phase existence range of the composition a) according to aspects of the invention (see FIG. 3). Particularly preferred NP-MCAs result in an enlargement of the temperature range ΔT of from 10% to 1000%, quite particularly preferably from 20% to 500%. The temperature range ΔT can be influenced in addition or alternatively to the enlargement of the surface area and/or the scale of the triangle.

By NP-MCAs are meant in particular molecules which consist of carbon, hydrogen and of at least one of the following types of atom (heteroatoms): silicon, oxygen, nitrogen, sulphur, phosphorus, fluorine, chlorine, bromine, iodine. Polar carbon atoms are preferably situated next to heteroatoms. Polar carbon atoms are not to be included in an alkyl chain or non-polar chain.

Preferred NP-MCAs within the meaning of aspects of the present invention comprise those that are selected from the group comprising alcohols, ketones, esters, heterocyclic compounds with 5 to 7 atoms per cycle, ethers, amides and amines, N-acylated amino acids, and some aldehydes which do not have a surfactant-like structure, thus do not have a directional head-to-tail structure. These are in particular alcohols (monoalcohols, dialcohols, trialcohols, etc.) which do not have a surfactant-like structure.

Advantageous, and therefore preferred, are those NP-MCA molecules of which the hydrophilic and hydrophobic areas are mixed in the molecule such that:

i) no terminal, non-polar chain which is situated on a primary or secondary carbon atom has 4 or more carbon atoms. Should the chain be longer, it must not account for more than 20% of the molecular weight;
ii) a non-polar chain that is intramolecular or situated on a tertiary carbon atom is not longer than 7 carbon atoms (in other words greater than for example 1,9-nonanediol) and accounts for more than 20% of the molecular weight. Larger chains are capable of remaining in the non-polar area, while the polar portions of the molecule are to be found in the hydrophilic area;
iii) in monocyclic alcohols, the shortest path through the cycle for the determination of the chain length after point i) and ii) is chosen as chain length;
iv) in polycyclic alcohols, only the completely non-polar cycles are taken into account for the determination of the chain length according to point i) and ii) and here the lowest number of carbon atoms is taken as chain length.

Because of the comparable polarity, what was said for alcohols applies analogously to amines and alcohol amines. The same applies analogously to fluorides, chlorides and molecules which are constructed from such groups.

A composition which comprises such non-structure-forming, mixed-chain structure amphiphiles from the group of the alcohols, amines and alcohol amines is also a subject of aspects of the present invention.

In particular ketones or acids and their weak salts and amides, as well as organyl sulphates and phosphates, can also be preferred NP-MCAs within the meaning of the present invention. Because of their slightly higher polarity compared with alcohols, a chain length increased by 1 applies here to terminal and intramolecular chains. Consequently, a composition which comprises such non-structure-forming, mixed-chain structure amphiphiles from the group of the ketones or acids and their weak salts and amides, as well as organyl sulphates and phosphates, is also a subject of aspects of the present invention.

Alkyl, alkenyl, alkinyl, aryl sulphides, phosphides and silicones/siloxanes can furthermore also be preferred NP-MCAs within the meaning of the present invention. Because of the lower polarity, a chain length reduced by 1 compared with alcohols applies here. As a result, a composition which comprises such non-structure-forming, mixed-chain structure amphiphiles with alkyl, alkenyl, alkinyl residues or from the group of the aryl sulphides, phosphides and silicones/siloxanes is also a subject of the present invention.

In addition, in particular NP-MCAs which contain several of the above-named functionalities are also preferred according to aspects of the invention, wherein different functional groups can also occur in the molecule. The chain lengths given in the case of alcohols here serve as chain lengths for delimiting conventional surfactant-like molecules, provided that the functionalities are not predominantly ketones, acids and their weak salts, amides or organyl sulphates or phosphates.

A preparation comprising an amphiphilic substance (NP-MCA) selected from the group consisting of alcohols, amines, alcohol amines, ketones, acids and their weak salts and amides, organyl sulphates and phosphates, alkyl, alkenyl, alkinyl residues, from the group of the aryl sulphides, phosphides and silicones/siloxanes is thus a preferred subject of the present invention.

Particularly preferred NP-MCAs are selected from diols of Formula I:

R₁R₂COH—(CH₂)ₙ—COHR₁R₂ [Formula I]
wherein
n can be=0, 1, 2, 3 or 4,
R₁ and R₂ are in each case independently of each other hydrogen or an unbranched or branched C₁-C₃ alkyl.

From this group in particular particularly preferred NP-MCAs are selected from the following diols: 1,3-propanediol, 1,3-butanediol, 1,4-butanediol, 1,5-pentanediol, 1,6-hexanediol, 2,3-butanediol, 2,4-pentanediol, 2-ethyl-1,3-hexanediol, 2,5-dimethyl-2,5-hexanediol, 2-methyl-2,4-pentanediol, 2-(n-butyl)-2-ethyl-1,3-propanediol or from 1,2-diols.

The named diols are suitable in particular for providing a preparation according to aspects of the invention.

Particularly preferred NP-MCAs are also selected from acetoacetates of Formula II:

C(R₃)₃—CO—CH₂—CO—O—R₄ [Formula II]
wherein
in each case independently of each other R₃ is hydrogen or a C₁ to C₂ alkyl and
R₄ is a branched or unbranched C₁ to C₄ alkyl;
   or from acetoacetates of Formula III:

CH₃—CO—CH₂—CO—O—R₅ [Formula III]
wherein
R₅ is a C₁ to C₄ alkyl.

From this group in particular particularly preferred NP-MCAs are selected from the following acetoacetates: ethyl acetoacetate, isopropyl acetoacetate, methyl acetoacetate, n-butyl acetoacetate, n-propyl acetoacetate or tert-butyl acetoacetate.

The named acetoacetates are suitable in particular for providing a preparation according to aspects of the invention.

Further preferred NP-MCAs are selected from diones of Formula IV

CH₃—(CH₂)p-CO—(CH₂)q-CO—(CH₂)r-CH₃ [Formula IV]
wherein
p, q, r independently of each other can be 0, 1 or 2, with the proviso that, if the sum of p, q and r=2, the compound according to Formula IV can also be cyclic (cyclohexanedione).

From this group in particular particularly preferred NP-MCAs are selected from the following diones: 2,3-butanedione (diacetyl), 2,4-pentanedione (acetylacetone), 3,4-hexanedione, 2,5-hexanedione, 2,3-pentanedione, 2,3-hexanedione, 1,4-cyclohexanedione or 1,3-cyclohexanedione.

The named diones are suitable in particular for providing a preparation according to aspects of the invention.

Likewise preferred NP-MCAs are selected from esters of Formula V

R₆—CO—O—R₇ [Formula V]
wherein
R₆ is a ring bond to R₇, CH₃ or COCH₃ and
R₇ is a (CH₂)₂—O-ring bond to R₆, (CH₂)₂—O—(CH₂)₃—CH₃, CH₂—CH₃ or CH₂—CH(CH₃)—O-ring bond to R₆.

From this group in particular particularly preferred NP-MCAs are selected from the following esters: (1-methoxy-2-propyl)-acetate, (2-butoxyethyl)-acetate, ethylene carbonate, ethyl pyruvate (2-oxo propanoic acid ethyl ester) or propylene carbonate.

The named esters are suitable in particular for providing a preparation according to aspects of the invention.

Further preferred NP-MCAs are selected from maleic or fumaric acid amides of Formula VI

R₈—CO—C═C—CO—O—R₉ [Formula VI]
wherein
R₈ is hydrogen, a branched or unbranched C₁-C₄ alkyl, or a branched or unbranched, linear or cyclic C₁-C₆ alkyl, wherein the C₁-C₆ alkyl is substituted by one or more groups selected from OH, NH₂, COOH, CO, SO₃H₂OP(OH)₂, and R₉ is hydrogen or a branched or unbranched C₁-C₄ alkyl.

From this group in particular particularly preferred NP-MCAs are selected from the following maleic acid amides and their methyl, ethyl, propyl and butyl esters: N-methylmaleamide; N-ethylmaleamide; N-(n-propyl)-maleamide; N-(i-propyl)-maleamide; N-(n-butyl)-maleamide; N-(i-butylmaleamide); N-(tert-butylmaleamide), as well as the corresponding fumaric acid amides and their methyl, ethyl, propyl and butyl esters.

Further preferred NP-MCAs are selected from: 2,2-dimethoxypropane, pyruvic aldehyde-1,1-dimethyl acetal, diacetane alcohol (2-methyl-2-pentanol-4-one), 2-butanol, 2-acetyl-gamma-butyrolactone, 3-amino-1H-1,2,4-triazole, gamma-butyrolactone, nicotinic acid amide, ascorbic acid, N-acetylamino acids, in particular N-acetylglycine, alanine, cysteine, valine or arginine, triethyl phosphate, n-butyl acetate, dimethyl sulphoxide or 2,2,2-trifluoroethanol.

According to aspects of the invention, the following NP-MCAs are quite particularly preferred, which are selected from the group consisting of ethyl acetoacetate; i-propyl acetoacetate; methyl acetoacetate; methyl isobutyrylacetate(methyl-(4-methyl-3-oxopentanoate)); n-butyl acetoacetate; n-propyl acetoacetate; tert-butyl acetoacetate; allyl acetoacetate; maleic acid amide(maleamic acid, maleamide), the following maleamides and their methyl, ethyl, propyl and butyl esters; N-methylmaleamide; N-ethylmaleamide; N-(n-propyl)-maleamide; N-(i-propyl)-maleamide; N-(n-butyl)-maleamide; N-(1-butylmaleamide); N-(tert-butylmaleamide); as well as the corresponding fumaric acid amides and their methyl, ethyl, propyl and butyl esters; 2,2-dimethoxypropane; diacetone alcohol (4-hydroxy-4-methylpentan-2-one); 1,3-butanediol; 1,4-butanediol; 1,5-pentanediol; 1,6-hexanediol; 2-ethyl-1,3-hexanediol, 2-methyl-2,4-pentanediol, 2-(n-butyl)-2-ethyl-1,3-propanediol; 1,3-propanediol; 2,3-butanediol; 2,4-pentanediol; 2,5-dimethyl-2,5-hexanediol; (1-methoxy-2-propyl)-acetate; (2-butoxyethyl)-acetate; 1,3-cyclohexanedione; 1,4-cyclohexanedione; 2,3-hexanedione; 2,3-pentanedione; 2,5-hexanedione; 3,4-hexanedione; acetylacetone (2,4-pentanedione, ACAC); diacetyl (2,3-butanedione); ethylene carbonate; propylene carbonate; 2-acetyl-gamma-butyrolactone; N-acetylcysteine and methyl, ethyl, propyl, butyl esters; N-acetylglutamic acid and methyl, ethyl, propyl, butyl esters; N-acetylglycine and methyl, ethyl, propyl, butyl esters; N-acetyltyrosine and methyl, ethyl, propyl, butyl esters; N-acetylvaline and methyl, ethyl, propyl, butyl esters; ethylpyruvate (2-oxopropanoic acid ethyl ester); pyruvic aldehyde-1,1-dimethyl acetal; 3-amino-1H-1,2,4-triazole; diethyl-3-oxoglutarate; diethylene glycol diethyl ether; diisopropyl ether; ethylene glycol diethyl ether; methylcarbamate; tert-butyl methyl ether; vinyl acetate; quinine (free base, as hydrochloride); adipic acid diamide; succinic acid imide; N-methylcaprolactam; acetic acid diethylamide; urea; thioacetamide; 1,2-phenylenediamine; 1,3-phenylenediamine; 1,4-diaminobutane; 1,4-diazabicyclo[2.2.2]octane; 1,4-phenylenediamine; 1,6-diaminohexane; 2-(4-methoxyphenyl)-ethylamine; 2-aminobenzamide; 2-aminophenol; dipropylamine; triethylamine; tyramine; anthranilic acid; DL-2-aminobutyric acid; serine; threonine; tyrosine; adipic acid; methylenesuccinic acid; trans-propene-1,2,3-tricarboxylic acid; cyclohexanol; cyclohexanone; dimedone (5,5-dimethylcyclohexane-1,3-dione); N,N-dimethylcyclohexylamine; trans-1,2-cyclohexanediol; (4-hydroxyphenyl)acetic acid; 1,3,5-trihydroxybenzene; 2-ethylpyridine; 2-methoxybenzoic acid; 2-methoxyphenol; 2-methylhydroquinone; 2-methylresorcinol; 2,4-dihydroxybenzoic acid; 2,6-dihydroxybenzoic acid; 3-aminophenol; 3,4-dihydroxybenzoic acid; 3,5-dihydroxybenzoic acid; 4-amino-3-nitrophenol; 4-aminophenol; 4-hydroxybenzaldehyde; 4-hydroxybenzoic acid; 5-methylresorcinol; acetylsalicylic acid; butylhydroxytoluene; N-phenyl-2,2′-iminodiethanol; N-phenylurea; methyl-, ethyl-, propyl-4-hydroxybenzoate; sulphanilic acid; vanillin; (2-ethoxyethyl)-acetate; (2-ethoxyethyl)-methacrylate; (2-hydroxypropoyl)-methacrylate; [2-(2-butoxyethoxy)-ethyl]-acetate; 1,2-propylene glycol diacetate; diethyl malonate; dimethyl acetylsuccinate; dimethyl carbonate; dimethyl fumarate; dimethyl glutarate; dimethyl malonate; ethyl acetate; ethylene glycol diacetate; ethyl formate; ethyl lactate; glycerol triacetate; isopropenyl acetate; methyl formate; methyl lactate; methyl propionate; propyl formate; propyl propionate; tetraethyl orthocarbonate; triethyl citrate; 1-benzylpiperidin-4-one; 1-cyclohexyl-2-pyrrolidone; 1H-benzotriazole; 2-aminothiazole; 2-ethoxy-3,4-dihydro-2H-pyran; 2-ethylpiperidine; 2-mercapto-1-methylimidazole; 2-methyltetrahydrofuran; 2,2,6,6-tetramethyl-4-piperidinol; ascorbic acid; caffeine, theobromine, theophylline and the corresponding ethylxanthines; coumarin-3-carboxylic acid; ectoine; hydroxyproline; imidazole; indole; indole-3-acetic acid and its salts; melamine (2,4,6-triamino-1,3,5-triazine); methyl nicotinate; ethyl nicotinate, nicotinamide; nicotinic acid; pyridine-2-carboxylic acid; pyridine-2,3-dicarboxylic acid; pyridine-4-carboxylic acid; tropine (3-tropanol); tryptamine; nitroethane; nitromethane; 2-methyl-1-butanol; isobutanol (2-methyl-1-propanol); tert-amyl alcohol; 1,3-cyclopentanedione; 2,6-dihydroxyacetophenone; 3-methyl-3-penten-2-one; acetophenone; diethyl ketone; dihydroxyacetone; ethyl methyl ketone; isobutyl methyl ketone (methyl isobutyl ketone, MIBK); isopropyl methyl ketone; methyl propyl ketone; propiophenone; 2-butanoxim; sulphanilamide; 1,2,6-hexanetriol; 2-[4-(2-hydroxyethyl)-1-piperazinyl]-ethanesulphonic acid; 2-amino-2-methyl-1,3-propanediol (AEPD, ammediol), individually or as a mixture including their derivatives.

The NP-MCA is preferably contained in the composition a) according to aspects of the invention at a level of 1-80 wt.-% relative to the total weight of composition a), particularly preferably of 2-25 wt.-%, quite particularly preferably of 10-24 wt.-%.

For the purpose of the present invention, oils are meant by the at least one water-insoluble substance with a solubility in water of less than 4 g per litre. The term oil denotes all hydrophobic substances which do not mix homogeneously with water or a watery liquid and form a separate phase. As some oils still largely dissolve in water, a water solubility of less than 4 grams per litre is additionally defined here. Preferably, the water-insoluble substances are those with a water solubility of less than 2 g per litre. These include e.g. alkanes (benzines) and cycloalkanes (preferably cyclohexane). Aromatics such as toluene, xylenes or other alkyl benzenes as well as naphthalenes also come into consideration.

Long-chain alkanoic acid esters, such as fatty oils and fatty acid alkyl esters or fatty alcohol ethers are preferred. According to aspects of the invention, benzyl acetate also belongs to the water-insoluble substances used. However, terpenes, e.g. monocyclic monoterpenes with cyclohexane structure, can also be used. Here terpenes from citrus fruits, such as lemon and/or orange terpenes or the limonene contained therein are particularly preferred. The water-insoluble substances a1) are preferably contained at a level of 0.1-90 wt.-% in the composition a) according to aspects of the invention, preferably of 0.5-75 wt.-%, particularly preferably of 1.0 to 50 wt.-%, quite particularly preferably of 1.5-30 wt.-% relative to the total weight of the composition a) according to aspects of the invention.

Higher alcohols for example can be used as further amphiphilic substances with surfactant structure. Particularly preferred here are above all cosurfactants with hydrophilic-lipophilic molecular proportions such as e.g. the n- and i-isomers of butanol, pentanol, hexanol, heptanol, octanol, nonanol, decanol, undecanol and dodecanol.

Cycloalkanols, such as cyclohexanol or particularly preferably phenyl alcohols such as phenyl methanol (benzyl alcohol), 2-phenylethanol and 3-phenyl-1-propanol are also preferred. Short-chain fatty acids, such as hexanoic, heptanoic, octanoic acids and their alkali or ammonium salts can also preferably be used. Their salts of ethanolamines are particularly preferred.

The further amphiphilic substances with surfactant structure are preferably contained at a level of from 2 to 45 wt.-% in the composition according to aspects of the invention, relative to the total weight of the composition a) according to aspects of the invention, particularly preferably from 2 to 40 wt.-%.

Particularly preferably, the further amphiphilic substance with surfactant structure has a water solubility of from 2 g to 128 g per litre and is selected from the group comprising C₄-C₁₂ alcohols, cycloalkanols, phenyl alcohols, short-chain fatty acids or their alkali or ammonium salts.

The composition a) according to aspects of the invention further comprises as component a3) anionic, cationic, amphoteric and/or non-ionic surfactants. Some preferably suitable surfactants are named in the following list.

As anionic surfactants, e.g. alkali or ammonium salts of long-chain fatty acids, alkyl(benzene)sulphonates, paraffin sulphonates, bis(2-ethylhexyl) sulphosuccinate, alkyl sulphates, such as above all sodium dodecyl sulphate and, for specific applications which involve e.g. corrosion protection, sometimes also alkyl phosphates (e.g. Phospholan® PE 65, Akzo Nobel) can be used.

As non-ionic surfactants, polyalkylene oxide-modified fatty alcohols, such as e.g. Berol® types (Akzo Nobel) and Hoesch T types (Julius Hoesch), as well as also corresponding octylphenols (Triton types) or nonylphenols, can be used A particular field of use is made possible by the heptamethyltrisiloxanes (e.g. Silwet® types, GE Silicones), as agents for greatly increasing the spraying properties of the liquids or for significantly lowering the interfacial tension.

As cationic surfactants, e.g. coco bis(2-hydroxyethyl-)methylammonium chloride or polyoxyethylene-modified talc methyl-ammonium chloride can be used. In addition, the use of suitable amphoteric surfactants is also possible, of which just betaines (cocoamidopropyl betaine) or sulphobetaines or sultaines (amidopropylhydroxysultaines) may be named by way of example from the known plurality. If a further pH range is to be covered, the coco dimethylamine oxide (Aromox® MCD, Akzo Nobel) has proved to be suitable.

The surfactants are contained in the composition a) according to aspects of the invention at a level of between 0.1 and 45 wt.-%, preferably between 1.0 and 30 wt.-%, quite preferably from 9.0 to 16.0 wt.-%, relative to the total weight of the composition a) according to aspects of the invention.

In addition, the invention relates to a method for producing the composition a) according to aspects of the invention. The method according to aspects of the invention for producing a composition a) according to aspects of the invention can be carried out by introducing at least one polar solvent in particular with hydroxy functionality, preferably in a quantity of between 1.0 and 90 wt.-%, relative to the complete composition, and dissolving an anionic, cationic, amphoteric and/or non-ionic surfactant, preferably in a quantity of from 0.1 to 45 wt.-%, relative to the complete composition, in this at 10 to 90° C. accompanied by stirring, adding water-insoluble substance(s), preferably in a quantity of from 0.1 to 90 wt.-%, relative to the complete composition, parallel to or after addition of surfactant and then converting the emulsion that has formed to an optically transparent enlarged microemulsion or a nanophase system by adding a further amphiphilic substance with surfactant structure and NP-MCA, preferably in a quantity of from 0.1 to 80 wt.-%, relative to the complete composition, and optionally adding excipients at the end of the mixing procedure.

The composition a) according to aspects of the invention is produced in particular by first introducing water or the solvent with hydroxy functionality into a suitable vessel and then dissolving the surfactant accompanied by stirring. In the process it is to be borne in mind that some surfactants may already contain water as supplied, with the result that the quantity of water precalculated in the formulation must be adjusted where necessary. When dissolving the surfactant, it must be ensured that the input of air into the solution is kept as small as possible in order to avoid excessive foaming. For industrial-scale implementation, there are already many variations of stirring units and stirrers for largely avoiding foaming. The stirring speed should not usually exceed 200 revolutions per minute when using propeller mixers and ideal ratios of stirrer diameter and container diameter. In addition, it must be borne in mind that some (concentrated) surfactants may form gels when water is added, which can make stirring and a further dispersion difficult. In such cases, the water-insoluble substances (oil phase) must, where necessary, be added first or parallel to the addition of surfactant. Foaming can also be prevented by subsequently adding the oil phase, as this often has a certain anti-foam action. After addition of the oil phase, a milky, cloudy emulsion has formed which clears due to the addition of the further amphiphilic substance with surfactant structure (for example alkanol), but at the latest after addition of the amphiphile without surfactant structure according to component a2) (for example an acetoacetate compound) and finally passes into an optically transparent enlarged microemulsion or a nanophase system. At the end, excipients and additives, such as for example thickeners (for example those from the group of the Aerosils) can also be added.

A subject of the invention is also a method for producing the composition a) according to aspects of the invention, according to which i) at least one polar solvent in particular with hydroxy functionality is introduced, ii) an anionic, cationic, amphoteric and/or non-ionic surfactant is dissolved in this at 10 to 90° C. accompanied by stirring, iii) water-insoluble substance(s) are added parallel to or after addition of surfactant and iv) the emulsion that has formed is then converted to an optically transparent microemulsion or a nanophase system by adding at least one NP-MCA and v) at the end of the preceding mixing procedure excipients are optionally added.

Preferably, at least one further amphiphilic substance with surfactant structure, for example a cosurfactant with hydrophilic-lipophilic molecular proportions, can be added to this mixture, in particular in the method steps i) and iv), preferably in the method steps ii) and iv).

During the mixing or after the mixing of the relevant components, a therapeutically, cosmetically or diagnostically active agent can be added in each phase of the forming composition or to the complete composition. Preferably in method steps ii), iii) and iv).

The quantity of the composition a) which, according to aspects of the invention, serves to enhance or improve the penetration or permeation of active ingredients into the skin or through the skin, can differ depending on the manner of the application and amount to between 0.01 wt. % and 100 wt. %, relative to the total weight of the ready-to-use preparation. In general, a person skilled in the art can ascertain by simple trial and error in what quantities the composition according to aspects of the invention is present in a complete preparation or product to be administered.

However, as a guide value it can be assumed that an improvement in the penetration and permeation of an active ingredient into the skin or through the skin in the range of from 0.5 to 90 wt. %, preferably 1.0 to 80 wt. %, in particular between 1.0 and 50 wt. %, relative to the total weight of the composition, is guaranteed.

A subject of the present invention is also a preparation which can be produced according to one of the methods described above.

A subject of the present invention is also the use of a preparation according to aspects of the invention for producing a pharmaceutical and cosmetic preparation for intradermal and transdermal application in humans and animals.

Concerning the quantity of an active ingredient to be incorporated into the composition according to aspects of the invention, this depends in particular on the nature of the active ingredient and can therefore vary greatly. A person skilled in the art in particular in the field of pharmacology, pharmacokinetics, dermatology or cosmetics will consequently carry out routine tests to find the dose in order to discover the optimum quantity of an active ingredient to be used for intradermal or transdermal application. A person skilled in the art will in these cases be led to achieve and maintain a desired blood level, which he can easily check by tests and methods know to him.

It can generally be assumed that the quantities of active ingredient can be contained in the composition according to aspects of the invention at a level of between 0.001 wt. % and 99.99 wt. %, relative to the total weight of the complete preparation.

In the case of active therapeutic active ingredients, such as for example tetracaine, quantities between 0.001% and 10.0 wt. %, preferably between 0.01 and 5.0 wt. %, particularly preferably between 0.05 and 3.0 wt. %, in particular between 0.1 and 2.0 wt. %, relative to the ready-to-use preparation, can be assumed.

In the case of the substance indigo carmine which is pharmacologically active or active in the circulatory system (J Tarnow et al., Klin. Wschr. 52: 506-508, 1974), quantities between 0.001 wt. % and 5.0 wt. %, preferably 0.005 wt. % and 1.0 wt. %, in particular between 0.01 wt. % and 0.1 wt. %, relative to the ready-to-use preparation, can be assumed.

In the case of active cosmetic substances, such as for example dyes, in particular tattoo dyes, quantities between 0.001 wt. % and 5.0 wt. %, preferably 0.005 wt. % and 2.5 wt. %, in particular 0.01 wt. % and 1.0 wt. %, relative to the ready-to-use preparation, can be assumed.

In the case of self-tanning cosmetic substances, so-called “self-tanning agents”, such as for example N-decanoyl-tyrosine salts (for example Tyrostan® from Sinerga S.p.a.,), quantities between 0.1 wt. % and 50 wt. %, preferably between 0.5 wt. % and 25 wt. %, in particular between 1.0 wt. % and 20.0 wt. %, can serve as a guideline.

For example with regard to skin lighteners, such as for example kojic acid or hydroquinone or azelaoyl glycine (Azeloglicina® from Sinerga S.p.a.), quantities between 0.1 and 2.0 wt. % or between 1.0 and 5.0 wt. %, relative to the ready-to-use preparation, can be assumed.

Another subject of the present invention is therefore a method for producing a preparation, according to steps in which i) at least one polar protic solvent, in particular with hydroxy functionality, is introduced, preferably in a quantity of between 1.0 and 90 wt.-%, relative to the complete preparation, ii) an anionic, cationic, amphoteric and/or non-ionic surfactant, preferably in a quantity of from 0.1 to 45 wt.-%, relative to the complete composition, is then dissolved in i) at 10 to 90° C. accompanied by stirring, iii) water-insoluble substance(s) are added, preferably in a quantity of from 0.1 to 90 wt.-%, relative to the complete preparation, parallel to or after addition of surfactant according to step ii), iv) the emulsion that has formed is then converted to an optically transparent nanophase system by adding at least one amphiphilic substance NP-MCA, preferably in a quantity of from 0.1 to 80 wt.-%, relative to the complete preparation, v) at the end of the mixing procedure comprising steps i) to iv) excipients are optionally added, vi) during or after mixing components i) to v) at least one therapeutically, cosmetically or diagnostically active agent is added and mixed.

It is advantageous if the pH of the active ingredient in the preparation, and optionally the preparation itself, lies in a range which on the one hand is not disadvantageous for the active ingredient and its stability, and on the other hand is identical to the physiological environment and state of the skin, or is at least so close to it that no incompatibilities with the skin or other skin irritations occur. It is therefore advantageous if the pH of the agent or the ready-to-use preparation varies within the slightly acid to neutral region.

The incorporation of the relevant active ingredient into the composition according to aspects of the invention can take place in a manner known per se by mechanical mixing or by stirring into the composition according to aspects of the invention. The mixing advantageously continues until a homogeneous or single-phase state has been achieved. The temperature to be observed during the mixing method depends on the stability of the agent. It can generally be assumed that the temperature during the mixing method can lie between 0° C. (ice bath) and room temperature (20° C.-25° C., for example 22° C.).

It is however also advantageously possible that the composition according to aspects of the invention is first applied to the skin and only then, temporally and spatially separated, is the desired active ingredient applied to the thus-prepared or pretreated area of skin. The period of time between the application of the composition and the application of the active ingredient depends on whether the composition is present in a still active quantity on the relevant area of skin, which depends on the skin type, the ambient temperature and on whether the area of skin to which the composition was applied is mechanically stressed by articles of clothing.

It can generally be assumed that the active ingredient can be applied immediately or several hours after the previous application of the composition according to aspects of the invention to the skin. The application of the active ingredient immediately after the application of the composition is in particular advantageous and advisable at higher temperatures of more than 20° C. (for example between 25° C. and 40° C.) and/or low relative atmospheric humidity of for example less than 50% (for example 5-30% relative humidity) and/or in the case of mechanical stress of the relevant part of the body by articles of clothing, wigs, prostheses or by similar items covering the body.

It can however be assumed that the relevant therapeutically or cosmetically active agent is applied immediately after the previous application of the composition according to aspects of the invention to the skin, which could comprise approximately a period of 1 to 60 seconds, up to 5 hours later, in particular up to 2 hours, quite particularly up to 30 minutes later, preferably within 10 minutes.

In the case of a diagnostically active agent, this period of time can also amount to days, depending on how and to what extent a desired reaction, for example an immunologically determined reaction, occurs in a diagnostically secure manner.

Such a method, according to which the composition according to aspects of the invention and the relevant therapeutically, cosmetically or diagnostically active agent provided for the intradermal or transdermal application is applied consecutively, is therefore also a subject of the present invention.

A subject of the present invention is also the use of the composition according to aspects of the invention for the consecutive, intradermal or transdermal application of a therapeutically, cosmetically or diagnostically active agent.

Accordingly it is advantageous if a composition according to aspects of the invention in combination with a therapeutically, cosmetically or diagnostically active agent is provided in spatially separated packaging units, in particular as a kit-of-parts, advantageously in an already therapeutically, cosmetically or diagnostically active, pre-metered quantity for a specific therapeutic or non-therapeutic intended use.

An agent or a pack comprising a kit-of-parts, containing a composition according to aspects of the invention spatially or physically separated in functional combination with a therapeutically, cosmetically or diagnostically active agent, is also a subject of the present invention.

A subject of the present invention is also a method for the non-therapeutic treatment of the human and animal body according to which a composition according to aspects of the present invention is mixed with a cosmetically active agent and is then applied to the skin of the relevant human or animal body together with it, or is not mixed with the cosmetically active agent and is applied separately and consecutively to the skin of the relevant human or animal body.

It is of course clear that, on the basis of the description of the present invention, a method for the therapeutic application of the preparation according to aspects of the invention can also advantageously be carried out, according to which a preparation according to aspects of the present invention is applied externally to the skin of a human or an animal requiring corresponding medication.

Alternatively it may be advantageous that the composition according to aspects of the invention is first applied externally to the skin of the human or animal, and the therapeutic agent is then applied to the thus pre-treated or prepared area of skin of the human or animal requiring corresponding medication.

The present invention furthermore relates to a method for achieving an improved, i.e. enhanced, intradermal or transdermal, preferably therapeutic, in particular therapeutic systemic, effect of an active ingredient applied to the skin, wherein this method consists of producing a preparation or composition according to aspects of the invention and applying this to selected areas of skin and optionally subsequently, i.e. consecutively, determining the percutaneously absorbed quantities of active ingredient according to methods and processes known per se.

Where the method comprises the treatment of an undesired or pathological, in particular local, condition of the skin, a preparation according to aspects of the invention containing a suitable dermatologically active agent can be applied to the area of skin requiring treatment in order to treat for example acne, keratoses, epithelial cell carcinoma, atopic dermatitis, psoriasis, infections and tumours caused by microorganisms, higher fungi (for example athlete's foot or trychophyton infections) and by viruses (for example warts or papilloma), and irritations or intolerance reactions or allergic reactions caused by medicinal products.

In addition, an essential intended use and an essential method in the context of the present invention is also to apply a locally or systemically active analgesic or anaesthetic to the skin, according to which a suitable analgesic or anaesthetic, for example tetracaine, is mixed with a composition according to aspects of the invention and is then applied, or, in consecutive manner, the composition according to aspects of the invention is applied to the skin first and the active ingredient is applied subsequently, as already described in detail.

So-called photodynamic tumour therapy, which is carried out by using suitable therapeutically active agents easily penetrating the skin, for example 5-aminolaevulic acid, is also a suitable method of use and procedure within the scope of the present invention.

In addition to the use of a therapeutically or cosmetically active agent, a suitable diagnostically active agent, for example an allergen or an indicator substance, can also be advantageously used in a way similar to that described for improved transportation into the skin or through the skin. For example in the context of a skin-allergy test or sensitization test (for example prick tests, intradermal tests, friction tests, epicutaneous tests, ROAT (repeated open application test) tests) one or more antigens can be applied to the skin together with the composition according to aspects of the invention or consecutively, as already described, in order to trigger a desired immune reaction. It is advantageous that there is no need to wound the skin as happens in the methods of the state of the art.

The quantity of the diagnostic agent to be applied essentially depends on the nature of this agent and can be determined by a person skilled in the art within the context of routine tests. However, as it can as a rule be assumed that these agents already act in small quantities, the application of quantities of between 0.0001 wt. % and 5.0 wt. %, preferably between 0.0005 wt. % and 1.0 wt. %, in particular between 0.001 wt. % and 0.1 wt. %, relative to the complete preparation, can serve as a guideline.

To this extent a subject of the present invention is also the use of the preparation according to aspects of the invention and of the composition for producing a preparation for diagnostics in humans and animals.

Comparable diagnostic methods thus also open up in a completely analogous manner to a therapeutic or cosmetic treatment.

A method for the diagnostic treatment of a human and animal is thus namely advantageously made possible, wherein this method consists of producing a preparation according to aspects of the invention containing a suitable diagnostically active agent and applying the latter to selected areas of skin.

Another method consists of producing a composition according to aspects of the invention and applying the latter to selected areas of skin and subsequently, i.e. consecutively, applying the diagnostic agent to the thus-prepared area of skin, and then determining and evaluating the skin reaction caused by the diagnostic agent used using methods and processes known per se.

A subject is therefore also a preparation according to the present description or as defined in the claims which, in addition to the relevant composition a), comprises at least one therapeutically, cosmetically or diagnostically active agent b) in a therapeutically, cosmetically or diagnostically active quantity.

The preparation according to aspects of the invention transports the relevant therapeutically, cosmetically or diagnostic active agent intradermally or transdermally, with no need for special further excipients. However, it is advantageously also possible to use a covering layer which can have a protective effect or act as a means for measured dosage by delaying the release of active ingredient, for example in the form of a film, plaster or dressing into which the preparation according to aspects of the invention has been incorporated or which is placed over the already applied preparation. Such plasters, preferably provided with a glue or adhesive material, are known to a person skilled in the art and can for example consist of woven fabric, polyethylene or latex. The use of such covering layers is advantageous if the preparation applied is to be protected against evaporation or mechanical stress due to, for example, articles of clothing or if the active, agent is to act over a prolonged period, for example days.

As regards the application forms and galenic formulations suitable for the preparation according to aspects of the invention, all the types and forms known to a person skilled in the art come into consideration, provided they do not adversely affect the mode of operation or are incompatible with one of the types of active ingredient to be applied.

Generally all known hypoallergenic formulations which can be used in humans and animals, for example creams, lotions, gas-powered sprays or pump sprays, aerosols, gels, ointments, suppositories or encapsulations, which are known to a person skilled in the art to be suitable for percutaneous application can be used.

### Therapeutically Active Agents

It is clear to a person skilled in the art that, in the case of the therapeutically active ingredients or agents which are to be administered transdermally, only those which he knows to be systemically active medicinal products, and which he can assume to be predominantly absorbed by the bloodstream without having a harmful effect are selected.

A person skilled in the art is also aware that, in the case of therapeutically active ingredients or agents to be administered intradermally, only those which he knows to be non-systemically active medicinal products and which he knows to develop their action predominantly at the site of application without having a harmful effect are selected.

A person skilled in the art can therefore select the therapeutically active agents known to him which are suitable for a percutaneous application and have a sufficient therapeutic potential from a broad spectrum for different indications and fields of use, in particular comprising antibiotics, antiviral active ingredients, anthelmintics, anti-inflammatory active ingredients, antipyretics, antiallergics and antihistamines, immunosuppressants, immunotherapeutics, antirheumatics and antiarthritics, antiasthmatics, antidepressives, antipsychotics, agents to combat diseases with neurological causes such as for example restless-leg syndrome, Parkinson's disease, Alzheimer's disease or spasms, antidiabetics, antihistamines, agents to combat pains including migraine, agents to combat malignant and benign tumours, agents to combat psoriasis, agents with a cardiovascular action including antiarrhythmics, calcium antagonists and betablockers, betamimetics, including tocolytics, muscle relaxants, hormones, antihypertensives, antidiuretics, vasodilatatory agents, blood-thinning agents, sedatives and tranquilizers, anaesthetics, in particular local anaesthetics (for example tetracaine), hypnotics including sleeping pills, opioid-type analgesics, in particular based on morphine, fentanyl or methadone compounds, non-opioid analgesics comprising nicotinergic and non-steroidal analgesics such as for example NSAIDs (non-steroidal anti-inflammatory drugs), and diagnostic agents in particular comprising skin-allergy tests or sensitization tests.

In particular the following active ingredients come into consideration for the preparation according to aspects of the invention, the use according to aspects of the invention or the method according to aspects of the invention: cocaine; local anaesthetics comprising aminoesters such as for example benozocaine, oxybuprocaine, procaine, tetracaine and aminoamides such as for example dibucaine, etidocaine, lidocaine, mepivacaine, prilocalne, bupivacaine, articaine, ropivacaine; analgesics comprising opioid analgesics for example morphine-, fentanyl or methadone-types and non-opioid analgesics comprising nicotinergic analgesics such as for example epibatidine; acid analgesics comprising NSAIDs (non-steroidal-anti-inflammatory drugs) such as for example salicylic acid derivatives, for example acetylsalicylic acid, methylsalicylic acid, diflunisal, phenylacetic acid derivatives such as for example diclofenac, 2-phenylpropionic acid derivatives such as for example ibuprofen or naproxen, oxicams such as for example meloxicam or piroxicam; non-acid analgesics such as for example 4-aminophenol derivatives such as for example paracetamol, pyrazolones such as for example metamizol or phenazone; antiphlogistics such as for example hydrocortisone acetate; tumour therapeutics such as for example 5-aminolevulinic acid; antimycotics such as for example terbinafine or clotrimazole; antihistamines such as for example ethylene diamines, for example mepyramine(pyrilamine), tripelennamine(pyribenzamine), antazoline, dimetindene, bamipine maleate, or ethanol amines such as for example diphenhydramine, carbinoxamine, doxylamine, clemastine, or alkyl amines such as for example pheniramine, chlorphenamine(chlorpheniramine), dexchlorphenamine, brompheniramine, triprolidine, or piperazines such as for example hydroxyzine and meclozine, or tricyclic antihistamines such as for example promethazine, alimemazine (trimeprazine), cyproheptadine and azatadine, wherein the antihistamines acrivastine, astemizole, cetirizine, ebastine, fexofenadine, loratadine, mizolastine, terfenadine or rupatadine are particularly recommended for systemic, transdermal use and the antihistamines azelastine, levocabastine, olopatadine or epinastine in particular for topical, intradermal application; further active ingredients that can be considered as suitable for the purpose according to aspects of the invention are for example those which are described for example in the U.S. Pat. No. 5,853,751 and U.S. Pat. No. 6,113,921, comprising acebutolol, acetaminophen, acetohydroxamic acid, acetophenazine, aciclovir, adrenocorticoids, allopurinol, alprazolam, aluminum hydroxide, amantadine, ambenonium, amiloride, amphotericin-B, potassium aminobenzoate, amobarbital, amoxicillin, amphetamine, ampicillin, androgens, anticoagulants, dione-type anticonvulsives, antithyroidal agents, appetite suppressants, aspirin, atenolol, atropine, azatadine, bacampicillin, baclofen, beclomethasone, belladonna, bendroflumethiazide, benzoyl peroxide, benzthiazide, benztropine, betamethasone, bethanechol, bifonazole, biperiden, bisacodyl, bromocriptine, bromodiphenhydramine, brompheniramine, buclizine, bumetanide, busulfan, butabarbital, butaperazine, caffeine, calcium carbonate, captopril, carbamazepine, carbenicillin, carbidopa and levodopa, carbinoxamine inhibitors, carbon anhydrase, carisoprodol, carphenazine, cascara, cefaclor, cefadroxil, cefalexin, cefradin, clofedianol, chloral hydrate, chlorambucil, chloramphenicol, chlordiazepoxide, chloroquine, chlorothiazide, chlorotrianisene, chlorpheniramine, chlorpromazine, chlorpropamide, chlorprothixene, chlortalidone, chlorzoxazone, colestyramine, cimetidine, cinoxacin, clemastine, clidinium, clindamycin, clofibrate, clomiphere, clonidine, clorazepate, cloxacillin, colchicine, coloestipol, conjugated oestrogen, contraceptives, cortisone, cromolyn, ciclacillin, cyclandelate, cyclizine, cyclobenzaprine, cyclophosphamide, cyclothiazide, cycrimine, cyproheptadine, danazol, danthron, dantrolene, dapsone, dextroamphetamine, dexamethasone, dexchlorpheniramine, dextromethorphan, diazepane, dicloxacillin, dicyclomine, diethylstilbestrol, diflunisal, digitalis, diltiazem, dimenhydrinate, dimethindene, diphenhydramine, diphenidol, diphenoxylate and atrophive, diphenylopyraline, dipyradamole, disopyramide, disulfuram, divalporex, docusate calcium, docusate potassium, docusate sodium, doxylamine, dronabinol ephedrine, epinephrine, ergoloid mesylate, ergonovine, ergotamine, erythromycin, esterified oestrogens, estradiol, estrogen, estron, estropipate, etharynic acid, ethchlorvynol, ethinylestradiol, ethopropazine, ethosaximide, ethotoin, fenoprofen, ferrous fumarates, ferrous gluconates, ferrous sulphate, flavoxate, flecamide, fluphenazine, fluprednisolone, flurazepam, folic acid, furosemide, gemfibrozil, gentamicine, glipizide, glyburide, glycopyrrolate, gold compounds, griseofulvin, guaifenesin, guanabenz, guanadrel, guanethidine, halazepam, haloperidol, hetacillin, hexobarbital, hydralazine, hydrochlorothiazide, hydrocortisone (cortisol), hydroflumethiazide, hydroxychloroquine, hydroxyzine, hyoscyamine, ibuprofen, indapamide, indomethacin, insulin, iofoquinol, iron polysaccharide, isoetharine, isoniazid, isopropamide, isoproterenol, isotretinoin, isoxsuprine, iodoxuridine, kaolin and pectin, ketoconazole, lactulose, levodopa, lincomycin, liothyronine, liotrix, lithium, loperamide, lorazepam, magnesium hydroxide, magnesium sulphate, magnesium trisilicate, maprotiline, meclizine, meclofenamate, medroxyprogesterone, melenamic acid, melphalan, mephenyloin, mephobarbital, meprobamate, mercaptopurine, mesoridazine, metaproterenol, metaxalone, methamphetamine, methaqualone, metharbital, methenamine, methicillin, methocarbamol, methotrexate, methsuximide, methyclothinzide, methylcellulose, methyldopa, methylergonovine, methylphenidate, methylprednisolone, methysergide, metoclopramide, metolazone, metoprolol, metronidazole, miconazole, minoxidil, mitotane, monamine oxidase inhibitors, nadolol, nafcillin, nalidixic acid, naproxen, narcotic analgesics, neomycin, neostigmine, niacin, nicotine, nifedipine, nitrates, nitroglycerine, nitrofurantoin, nomifensine, norethindrone, norethindrone acetate, norgestrel, nylidrin, nystatin, orphenadrine, oxacillin, oxazepam, oxprenolol, oxymetazoline, oxyphenbutazone, pancrelipase, pantothenic acid, papaverine, para-aminosalicylic acid, paramethasone, paregoric (“camphorated tincture of opium”, also known as “tinctura opii camphorata”), pemoline, penicillamine, penicillin, penicillin-v, pentobarbital, perphenazine, phenacetin, phenazopyridine, pheniramine, phenobarbital, phenolphthalein, phenprocoumon, phensuximide, phenylbutazone, phenylephrine, phenylpropanolamine, phenyltoloxamine, phenyloin, pilocarpine, pindolol, piperacetazine, piroxicam, poloxamers, calcium polycarbophil, polythiazide, potassium supplements, prazepam, prazosin, prednisolone, prednisone, primidone, probenecid, probucol, procainamide, procarbazine, prochlorperazine, procyclidine, promazine, promethazine, propantheline, propranolol, pseudoephedrine, psoralen, psyllium, pyridostigmine, pyridoxine, pyrilamine, pyrvinium, quinestrol (17a-ethynyl-1,3,5(10)-estratriene-3,17b-diol-3-cyclopentyl ether), quinethazone (Hydromox®), quinidine, quinine, ranitidine, rauwolfia alkaloids, riboflavin, rifampicin, ritodrine, salicylates, secopolamine, secobarbital, senna, sennosides a and b, simethicone, sodium bicarbonate, sodium phosphate, sodium fluoride, spironolactone, sucralfate, sulfacytine, sulfamethoxazole, sulfasalazine, sulfinpyrazone, sulfisoxazole, sulindac, talbutal, tamazepam, terbutaline, terfenadine, terpin hydrate, tetracycline, thiabendazole, thiamine, thioridazine, thiothixene, thyroglobulin, thyroid, thyroxine, ticarcillin, timolol, tocamide, tolazamide, tolbutamide, tolmetin, trazodone, tretinoin, triamcinolone, triamterene, triazolam, trichlormethiazide, tricyclic antidepressants, tridihexethyl, trifluoperazine, triflupromazine, trihexyphenidyl, trimeprazine, trimethobenzamine, trimethoprim, tripelennamine, triprolidine, tyrosine derivatives such as for example melanin or L-dopa (L-3,4-dihydroxyphenylalanine), valproic acid, verapamil, vitamin A, vitamin B-12, vitamin C, vitamin D, vitamin E, vitamin K, xanthines as well as amphotericin B, barbiturates, diphenoxylate, prostaglandin, retinoid or carotene.

These active ingredients named here by way of example, which are in no way to be regarded as a complete list of the therapeutic agents that are known to a person skilled in the art and suitable, can be used according to the present invention individually or in combination, depending on the medical indication.

### Cosmetically Active Agents

It is known to a person skilled in the art that, in the case of cosmetically active ingredients or agents to be administered to the skin or intradermally, only those which he knows to be non-systemically active, not to have a property equated with or acting in the same way as a medicinal product and to develop their cosmetic effect predominantly at the site of application, without having a harmful effect, are to be selected.

In connection with the use of a cosmetically active agent the term “intradermal” or “transdermal” is to be understood more specifically to mean the site of action of the cosmetic agent. Consequently, an intradermal transportation of the cosmetic agent covers the development of the cosmetic effect preferably at the dermal layer below the stratum corneum, as is the case for example with skin lightening, tattooing or the cosmetic treatment of acne. In the case of a transdermal transportation of a cosmetic active ingredient, deeper-lying dermal layers are preferably affected, as is the case for example in the cosmetic treatment of cellulite.

A person skilled in the art can therefore select the cosmetically active agents known to him which are suitable for a corresponding application and have a sufficient cosmetic potential from a broad spectrum of different application areas, in particular comprising skin colouring agents, such as for example tattoo pigments, self-tanning agents, UV filters, skin lighteners including agents to combat hyperpigmentation and age spots, anti-aging agents, anti-cellulite agents or anti-acne agents, agents to combat dandruff as well rough and dry skin.

In particular, for the preparation according to aspects of the invention, the use according to aspects of the invention or the method according to aspects of the invention, the following cosmetic active ingredients come into consideration for example: dihydroxyacetone, erythrulose, tyrosine, afamelanotide (melanotan I), kojic acid, kojic aminopropyl phosphate, tyrosine derivatives such as for example melanin or L-dopa (L-3,4-dihydroxyphenylalanine), hydroquinone, tyrostan, vitamins of the B group such as for example thiamine (vitamin B1), riboflavin (vitamin B2), nicotinic acid and nicotinic acid amide (vitamin B3), pantothenic acid and panthenol (vitamin B5) including their derivatives, in particular their esters and ethers as well as the cationically derivatized panthenols and pantolactone, pyridoxine and pyridoxal as well as pyridoxamine (vitamin B6); 1-ascorbic acid (vitamin C), 1-ascorbic phosphate, 1-ascorbic glucoside, vitamin E vitamins of the F group or essential fatty acids such as for example linoleic acid, linolenic acid or arachidonic acid, eicosapentanoic acid; biotin or (3aS,4S,6a R)-2-oxohexahydrothienol[3,4-d]-imidazole-4-valeric acid (vitamin H), plant extracts and distillates such as for example from green tea, oak bark, stinging nettle, witch hazel, hops, camomile, burdock root, horsetail, whitethorn, lime blossom, almonds, aloe vera, spruce needle, horse chestnut, sandalwood, cedar, coconut, mango, apricot, lime, wheat, kiwi, melon, orange, grapefruit, sage, rosemary, birch, hollyhock, cuckoo flower, heath milkwort, yarrow, thyme, balm, common restharrow, coltsfoot, marshmallow, meristem, ginseng and stem ginger, as can be obtained in known manner from petals or the green leaves of the relevant plants by means of extraction with water or low, mono- or polyhydric alcohols (ethanol, isopropanol, ethylene glycol or propylene glycol), arbutin, [cinnamic acid], botulinum toxins or urea.

These active ingredients named by way of example, which are in no way to be regarded as a complete list of the cosmetic agents that are known to a person skilled in the art and suitable, can be used according to the present invention individually or in combination, as required.

In order to test the action of the preparation according to aspects of the invention compared with active ingredient formulations without the composition a) according to aspects of the invention, in vitro tests can be carried out in per-se known manner on isolated human skin or on animal skin, for example isolated pig skin. This can be carried out by applying, preferably simultaneously, a preparation according to aspects of the present invention and the comparison sample in per-se known manner to directly adjacent areas of one and the same skin preparation, or under standardized conditions and the permeation measured in respect of speed and/or transported quantity.

The following examples are intended to explain the invention in still more detail, without limiting it to same in any way.

### Example 1

### Preparation with Tetracaine Suitable for Transdermal Transportation

| | Quantity |
|---|---|
| Component | (wt. %) |
| Water | 35.70 |
| 2-ethyl-1,3-hexanediol¹⁾ | 17.87 |
| Triethylcitrate²⁾ | 6.89 |
| Cetiol OE³⁾ | 20.03 |
| Tetracaine hydrochloride⁴⁾ | 1.09 |
| Lutensol TO 3⁵⁾ | 11.14 |
| Tween 80⁶⁾ | 6.86 |
| Sodium cocoyl isethionate⁷⁾ | 0.42 |

¹⁾Fluka Sigma-Aldrich Chemie GmbH, Taufkirchen, Germany
²⁾KMF, VWR International GmbH, Dresden, Germany
³⁾Cognis GmbH, Düsseldorf, Germany
⁴⁾Fluka Sigma-Aldrich Chemie GmbH, Taufkirchen, Germany
⁵⁾BASF SE, Ludwigshafen, Germany
⁶⁾AppliChem GmbH, Darmstadt, Germany
⁷⁾Elfan AT 84G, Akzo Nobel Surface Chemistry AB, SE 444 85, Sweden

The water was introduced at room temperature (22° C.) and the components 2-ethyl-1,3-hexanediol, triethylcitrate, Cetiol OE, Lutensol TO 3 and sodium cocoyl isethionate added immediately after one another and stirred. Tween 80 was then added to this solution accompanied by gentle stirring until a single homogeneous phase was achieved. The tetracaine hydrochloride was then stirred into this single-phase mixture until a single-phase state was again achieved. A single-phase fluid nanophase system formed. Following application of the tetracaine-containing nanophase fluid to the inside of the armpit, a transdermal anaesthetic action, which did not occur without the nanophase fluid, was able to be ascertained at the site of the application.

### Example 2

### Composition Suitable for Transdermal Transportation

| | Quantity |
|---|---|
| Component | (wt. %) |
| Water | 36.02 |
| 2-ethyl-1,3-hexanediol | 18.12 |
| Triethylcitrate | 7.01 |
| Cetiol OE | 20.21 |
| Lutensol TO 3 | 11.29 |
| Tween 80 | 6.94 |
| Sodium cocoyl isethionate | 0.40 |

The water was introduced at room temperature (22° C.) and the components 2-ethyl-1,3-hexanediol, triethylcitrate, Cetiol OE, Lutensol TO 3 and sodium cocoyl isethionate added immediately after one another and stirred. Tween 80 was then added to this solution accompanied by gentle stirring until a single homogeneous phase was achieved. A single-phase fluid nanophase system formed.

Such a composition is also suitable for a consecutive application according to which, after the application of this composition, an active ingredient can be subsequently applied to the area of skin thus prepared.

### Example 3

### Preparation with Tetracaine Suitable for Transdermal Transportation

| | Quantity |
|---|---|
| Component | (wt. %) |
| Water | 32.42 |
| Ethyl acetoacetate⁸⁾ | 24.79 |
| Cetiol OE | 15.58 |
| Tetracaine hydrochloride | 1.01 |
| Lutensol TO 3 | 19.77 |
| Sodium cocoyl isethionate | 6.43 |

⁸⁾Fluka Sigma-Aldrich Chemie GmbH, Taufkirchen, Germany

The water was introduced at room temperature (22° C.) and the components 2-ethyl-1,3-hexanediol, Cetiol OE, Lutensol TO 3 and sodium cocoyl isethionate added immediately after one another and stirred until a single homogeneous phase was achieved. Then tetracaine hydrochloride was stirred into this single-phase mixture until a single-phase state was again achieved. A single-phase fluid nanophase system formed. Following application of the tetracaine-containing nanophase fluid to the inside of the armpit, a transdermal anaesthetic effect could be ascertained at the site of the application, which did not occur without the nanophase fluid.

### Example 4

### Composition Suitable for Transdermal Transportation

| | Quantity |
|---|---|
| Component | (wt. %) |
| Water | 32.74 |
| Ethyl acetoacetate | 25.05 |
| Cetiol OE | 15.74 |
| Lutensol TO 3 | 19.97 |
| Sodium cocoyl isethionate | 6.50 |

The water was introduced at room temperature (22° C.) and the components ethylacetoacetate, Cetiol OE, Lutensol TO 3 and sodium cocoyl isethionate added immediately after one another and stirred. A single-phase fluid nanophase system formed.

Such a composition is also suitable for a consecutive application according to which, after the application of this composition, an active ingredient can be subsequently applied to the area of skin thus prepared.

### Example 5

### Composition with Tyrostan® for Tanning Suitable for Intradermal Transportation

| | Quantity |
|---|---|
| Component | (wt. %) |
| Water | 33.22 |
| Tyrostan ® | 8.43 |
| (N-decanoyltyrosine, potassium salt)⁹⁾ | |
| 2-ethyl-1,3-hexanediol | 16.64 |
| Triethylcitrate | 6.35 |
| Cetiol OE | 18.65 |
| Lutensol TO 3 | 10.36 |
| Tween 80 | 6.35 |

⁹⁾Sinerga, Pero, Italy

The water was introduced at room temperature (22° C.) and the components 2-ethyl-1,3-hexanediol, triethylcitrate, Cetiol OE and Lutensol TO 3 added immediately after one another and stirred. Tween 80 was then added to this solution accompanied by gentle stirring until a single homogeneous phase was achieved. Tyrostan® was then stirred into this single-phase mixture until a single-phase state was again achieved. A single-phase fluid nanophase system formed.

Following application of the Tyrostan®-containing nanophase fluid to the inside of the armpit, an intradermal bronzing action, which did not occur without the nanophase fluid, was able to be ascertained on the skin at the site of the application.

### Example 6

### Composition with Indigo Carmine Suitable for Intradermal Transportation

| | Quantity |
|---|---|
| Component | (wt. %) |
| Water | 36.79 |
| Ethyl acetoacetate | 9.19 |
| 3-methoxy-1-butanol¹⁰⁾ | 9.18 |
| Cetiol OE | 18.30 |
| Indigo carmine¹¹⁾ | 0.05 |
| Lutensol TO 3 | 13.73 |
| Tween 80 | 12.76 |

¹⁰⁾ACROS, Fisher Scientific GmbH, Nidderau, Germany
¹¹⁾ACROS, Fisher Scientific GmbH, Nidderau, Germany

The water was introduced at room temperature (22° C.) and the components ethyl acetoacetate, 3-methoxy-1-butanol, Cetiol OE and Lutensol TO 3 added immediately after one another and stirred. Tween 80 was then added to this solution accompanied by gentle stirring until a single homogeneous phase was achieved. The indigo carmine was then stirred into this single-phase mixture until a single-phase state was again achieved. A single-phase fluid nanophase system formed.

Following application of the indigo carmine-containing nanophase fluid to the inside of the armpit, an intradermal blue-colouring action, which did not occur without the nanophase fluid, was able to be ascertained on the skin at the site of the application.

### Example 7

### Composition Suitable for Intradermal Transportation

| | Quantity |
|---|---|
| Component | (wt. %) |
| Water | 36.76 |
| Ethyl acetoacetate | 9.19 |
| 3-methoxy-1-butanol¹⁰⁾ | 9.19 |
| Cetiol OE | 18.38 |
| Lutensol TO 3 | 13.73 |
| Tween 80 | 12.76 |

¹⁰⁾ACROS, Fisher Scientific GmbH, Nidderau, Germany
¹¹⁾ACROS, Fisher Scientific GmbH, Nidderau, Germany

The water was introduced at room temperature (22° C.) and the components ethyl acetoacetate, 3-methoxy-1-butanol, Cetiol OE and Lutensol TO 3 added immediately after one another and stirred. Tween 80 was then added to this solution accompanied by gentle stirring until a single homogeneous phase was achieved. A single-phase fluid nanophase system formed. Such a composition is also suitable for a consecutive application according to which, after the application of this composition, an active ingredient can be subsequently applied to the area of skin thus prepared.

### Example 8

### Application of a Dye-Containing Fluid Nanophase System

A dye-containing nanophase fluid according to Example 6 was compared with an aqueous solution with the same dye concentration (0.05 wt. % indigo carmine). For this purpose the skin on the inside of the test subject's armpit was treated with the aqueous solution of indigo carmine (indigo sulphonic acid, Na salt) and the nanophase fluid with indigo carmine. For this, the liquids were applied to the skin in special patterns, such as for example in the form of rhombi, dots and dashes. The chronology of the fading on the skin was documented photographically. After 8 hours both sites were washed with a wet cloth in the same operation and the effect of washing photographically documented on the basis of the fading of the colour. It was striking that the aqueous system initially coloured the surface of the skin more rapidly and more intensively blue than the nanophase fluid. However, in this comparative test the colouring persisted only at the surface, and mainly penetrated hair pores and small creases, with the result that, after the solution dried, these were more strongly coloured than higher layers of skin. Despite the striking colouring, at the end of the test period the dye did not remain on the skin after washing, apart from a few spots of colour in deeper hair pores.

On the other hand, in the case of the nanophase fluid the colouring was more uniform, i.e. the difference in colour intensity between wrinkles and creases and smooth areas of skin was slight. A strong colouring of the uppermost layers of skin was not able to be observed. After washing, visible and clear coloured patterns remained. Microscopic examination no longer revealed any colouring directly on the skin, or in creases or hair pores, but in deeper layers of skin beneath the stratum corneum, with the result that the patterns were similar to tattoos. The coloured patterns disappeared within another 48 hours due to the dye being taken away in the bloodstream. Consequently an intradermal transport of the dye took place. The occurrence of the dye or its decomposition products in the blood was not investigated.

### Example 9

### Detection of the Fluid Nanophase Systems

Green laser-beam scatter experiments (Conrad Electronic, Germany, model no. GLP-101, 530-545 nm) for detecting the nanostructuring in nanophase systems. The results are shown in FIG. 1:

a) Water: Green laser beam is not visible in the liquid, i.e. no scatter and therefore no nanostructuring.
b) Composition according to aspects of the invention consisting of:
Aqueous phase: water (35.70 wt. %); oil phase: Cetiol OE (20.03 wt. %); surfactant: Lutensol TO 3 (11.14 wt. %), Tween 80 (6.86 wt. %), sodium cocoyl isethionate (0.42 wt. %); NP-MCA: triethylcitrate (6.89 wt. %), 2-ethyl-1,3-hexanediol (17.87 wt. %), active ingredient: tetracaine hydrochloride (1.09 wt. %); the respective percentages by weight given are relative to the complete composition.

The green laser beam is visible due to scattering, in other words the liquid is nanostructured. Moreover, a red laser beam is barely scattered, as here the wavelength of the red light is too large for an interaction.

1. Preparation for external application in humans and animals, comprising in combination:
a) a composition in the form of a fluid nanophase system comprising the components
a1) at least one water-insoluble substance with a water solubility of less than 4 grams per litre, in a quantity of from 0.1 to 90 wt.-%;
a2) at least one amphiphilic substance NP-MCA which does not have a surfactant structure, is not structure-forming on its own, the solubility of which in water or oil is between 4 g and 1000 g per litre and which preferably does not accumulate at the oil-water interface, in a quantity of from 0.1 to 80 wt.-%;
a3) at least one anionic, cationic, amphoteric and/or non-ionic surfactant; in a quantity of from 0.1 to 45 wt.-%;
a4) at least one polar protic solvent, in particular with hydroxyl functionality, in a quantity of between 1.0 and 90 wt.-%; and
a5) optionally one or more excipients, in a quantity of from 0.01 to 10 wt.-%, wherein the given percentages are in each case relative to the total weight of the composition;
and
b) at least one therapeutically, cosmetically or diagnostically active agent in a therapeutically, cosmetically or diagnostically active quantity.

2. The preparation according to claim 1, wherein the amphiphilic substance NP-MCA according to component a2), when added to an oil-water-surfactant system containing the constituents a1), a3) and a4), and optionally a5) at 4 wt.-% relative to the total weight of the system, results in an at least 5% enlargement of the surface area of the triangle contained in the phase diagram represented in FIG. 3 which is determined by the three corner points:
i) the X-point;
ii) the upper intersection point of the boundary area between the single-phase and the two-phase area with the tangent laid parallel to the temperature Y-axis at the starting Lα-field; and
iii) the lower intersection point of the boundary area between the single-phase and the two-phase area with the tangent laid parallel to the temperature Y-axis at the starting Lα-field.

3. The preparation of claim 1, wherein the composition a) contains at least one further amphiphilic substance with surfactant structure.

4. The preparation of claim 1, wherein the amphiphilic substance (NP-MCA) is selected from the groups consisting of
a) diols of Formula I:
R₁R₂COH—(CH₂)ₙ—COHR₁R₂ [Formula I]
wherein
n can be =0, 1, 2, 3 or 4,
R₁ and R₂ are in each case independently of each other hydrogen or an unbranched or branched C₁-C₃ alkyl,
also comprising the diols: 1,3-propanediol, 1,3-butanediol, 1,4-butanediol, 1,5-pentanediol, 1,6-hexanediol, 2,3-butanediol, 2,4-pentanediol, 2-ethyl-1,3-hexanediol, 2,5-dimethyl-2,5-hexanediol, 2-methyl-2,4-pentanediol, 2-(n-butyl)-2-ethyl-1,3-propanediol or of 1,2-diols;
b) acetoacetates of Formula II:
C(R₃)₃—CO—CH₂—CO—O—R₄ [Formula II]
wherein
in each case independently of each other R₃ is hydrogen or a C₁ to C₂ alkyl and R₄ is a branched or unbranched C₁ to C₄ alkyl;
or acetoacetates of Formula III:
CH₃—CO—CH₂—CO—O—R₅ [Formula III]
wherein R₅ is a C₁ to C₄ alkyl;
also comprising the compounds ethyl acetoacetate, iso-propyl acetoacetate, methyl acetoacetate, n-butyl acetoacetate, n-propyl acetoacetate or tert-butyl acetoacetate;
c) diones of Formula IV
CH₃—(CH₂)p-CO—(CH₂)q-CO—(CH₂)r-CH₃ [Formula IV]
wherein
p, q, r independently of each other can be 0, 1 or 2, with the proviso that, if the sum of p, q and r=2, the compound according to Formula IV can also be cyclic,
also comprising the compounds diones: 2,3-butanedione (diacetyl), 2,4-pentanedione (acetylacetone), 3,4-hexanedione, 2,5-hexanedione, 2,3-pentanedione, 2,3-hexanedione, 1,4-cyclohexanedione or 1,3-cyclohexanedione;
d) esters of Formula V
R₆—CO—O—R₇ [Formula V]
wherein
R₆ is a ring bond to R₇, CH₃ or COCH₃ and R₇ is (CH₂)₂—O— ring bond to R₆, (CH₂)₂—O—(CH₂)₃—CH₃, CH₂—CH₃ or CH₂—CH(CH₃)—O— ring bond to R₆,
also comprising the compounds (1-methoxy-2-propyl)-acetate, (2-butoxyethyl)-acetate, ethylene carbonate, ethyl pyruvate (2-oxo propanoic acid ethyl ester) or propylene carbonate; and
e) maleic or fumaric acid amides of Formula VI
R₈—HN—CO—C═C—CO—O—R₉ [Formula VI]
wherein
R₈ is hydrogen, a branched or unbranched C₁-C₄ alkyl, or a branched or unbranched, linear or cyclic C₁-C₆ alkyl, wherein the C₁-C₆ alkyl is substituted by one or more groups selected from OH, NH₂, COOH, CO, SO₃H₂OP(OH)₂, and R₉ is hydrogen or a branched or unbranched C₁-C₄ alkyl,
also comprising the maleic acid amides and their methyl, ethyl, propyl and butyl esters comprising N-methylmaleamide; N-ethylmaleamide; N-(n-propyl)-maleamide; N-(1-propyl)-maleamide; N-(n-butyl)-maleamide; N-(1-butyl maleamide); N-(tert-butyl maleamide), as well as the corresponding fumaric acid amides and their methyl, ethyl, propyl and butyl esters, 2,2-dimethoxypropane, pyruvic aldehyde-1,1-dimethyl acetal, diacetanealcohol (2-methyl-2-pentanol-4-one), 2-butanol, 2-acetyl-gamma-butyrolactone, 3-amino-1H-1,2,4-triazole, gamma-butyrolactone, nicotinic acid amide, ascorbic acid, N-acetylamino acids, N-acetylglycine, alanine, cysteine, valine or arginine, triethyl phosphate, n-butyl acetate, dimethyl sulphoxide or 2,2,2-trifluoroethanol.

5. The preparation according to of claim 1, wherein the amphiphilic substance (NP-MCA) is selected from the acetoacetates of Formula III
CH₃—CO—CH₂—CO—O—R₅ [Formula III]
wherein R₅ is a C₁ to C₄ alkyl.

6. The preparation of claim 1, comprising an amphiphilic substance (NP-MCA) selected from the group consisting of alcohols, amines, alcohol amines, ketones, acids and their weak salts and amides, organyl sulphates and phosphates, alkyl, alkenyl, alkinyl residues, from the group of the aryl sulphides, phosphides and silicones/siloxanes.

7. The preparation of claim 1, wherein the amphiphilic substance (NP-MCA) is contained at a level of between 2 and 25 wt.-%, relative to the total weight of the composition.

8. A method for producing a preparation according to claim 1 comprising the steps of:
i) at least one polar protic solvent;
in particular with hydroxyl functionality, preferably in a quantity of between 1.0 and 90 wt.-%, relative to the complete preparation, is introduced;
ii) an anionic, cationic, amphoteric and/or non-ionic surfactant, preferably in a quantity of from 0.1 to 45 wt.-%, relative to the complete composition, is then dissolved in i) at 10 to 90° C. accompanied by stirring;
iii) water-insoluble substance(s), preferably in a quantity of from 0.1 to 90 wt.-%, relative to the complete preparation, is added parallel to or after addition of surfactant according to step ii);
iv) the emulsion that has formed is then converted to an optically transparent nanophase system by adding at least one amphiphilic substance NP-MCA, preferably in a quantity of from 0.1 to 80 wt.-%, relative to the complete preparation;
v) at the end of the mixing procedure comprising the steps i) to iv) excipients are optionally added; and
vi) during or after the mixing of components i) to v) at least one therapeutically, cosmetically or diagnostically active agent is added and mixed.

9. (canceled)

10. A method and for the intradermal or transdermal application of a therapeutically, cosmetically or diagnostically active agent in humans or animals, comprising the intradermal or transdermal application of the preparation of claim 1 to a human or animal.

11. A method for improving or enhancing the percutaneous penetration or permeation of an externally applied active ingredient or mixture of active ingredients, comprising percutaneous application of the preparation of claim 1 to a human or animal.

12. The method of claim 11, for increasing the penetration or permeation of an active ingredient into or through human or animal skin.

13. (canceled)

14. The method of claim 11, wherein the composition a) and the therapeutically, cosmetically or diagnostically active agent b) are applied consecutively or together.

15. A method and for producing a pharmaceutical or cosmetic preparation for intradermal or transdermal application comprising preparing the preparation of claim 1 in a form suitable for intradermal and transdermal applications in or to humans or animals.

16. A method for producing a preparation suitable for diagnosis in humans or animals, comprising preparing a preparation or a compositions a) according to claim 1 in a form suitable for diagnosis in humans or animals.

17. An agent or pack comprising a kit-of-parts containing the preparation of claim 1, wherein the composition a) is spatially or physically separated in functional combination with the therapeutically, cosmetically or diagnostically active agent b).

18. A method for the non-therapeutic treatment of the human and animal body, wherein the preparation of claim 1 is mixed with a cosmetically active agent and is then applied together to the skin of the relevant human or animal body, or is not mixed with the cosmetically active agent and is applied separately consecutively to the skin of the relevant human or animal body.
